(19) United States
(12) Patent Application Publication
(10) Pub. No.: US 2012/0238464 A1
(43) Pub. Date: Sep. 20, 2012

(54) Biomarkers for Predicting the Recurrence of Colorectal Cancer Metastasis

(75) Inventors: Minoru Koi, Dallas, TX (US); C. Richard Boland, Dallas, TX (US)

(73) Assignee: Baylor Research Institute, Dallas, TX (US)

(21) Appl. No.: 13/421,636

(22) Filed: Mar. 15, 2012

Related U.S. Application Data
(60) Provisional application No. 61/454,107, filed on Mar. 18, 2011.
(60) Provisional application No. 61/549,541, filed on Oct. 20, 2011.

(51) Int. Cl. C40B 30/04; C12Q 1/68; C40B 40/06

(52) U.S. Cl. 506/9; 506/16; 435/6.11

(57) ABSTRACT

The present invention includes biomarkers and methods for predicting recurrence-free survival and determination of risk for colorectal liver metastasis (LM) by determining a level of microsatellite instability at tetranucleotide repeats (EMAST) and at mono- and a dinucleotide repeat loci (MSI-L) or a SMARCA2R-LOH in colorectal cancer (CRC) patients. Results obtained indicate that stage II and III patients with MSI-M had a shorter recurrence-free survival than the rest of patients with high levels of MSI (MSI-H) or with highly stable microsatellites, and that MSI-M is an independent predictor for recurrent distant metastasis in primary stage II and III CRCs. It was found that SMARCA2R-LOH and MSI-M are found in stage IV primary CRC and LM tissues.

### CROSS-REFERENCE TO RELATED APPLICATIONS

This application claims priority to U.S. Provisional Application Ser. No. 61/454,107, filed Mar. 18, 2011, and U.S. Provisional Application Ser. No. 61/549,541, filed Oct. 10, 2011, the entire contents of each are incorporated herein by reference.

### STATEMENT OF FEDERALLY FUNDED RESEARCH

This invention was made with U.S. Government support under Contract Nos. R01CA72851 and CA 29286 awarded by the National Cancer Institute (NCI)/National Institutes of Health (NIH). The government has certain rights in this invention.

### TECHNICAL FIELD OF THE INVENTION

The present invention relates in general to primary colorectal cancers (CRCs). More particularly, the invention relates to markers for predicting the recurrence of distant metastasis of stage II and III primary CRCs and methods for identifying CRC patients at high risk for the recurrence of metastasis.

### REFERENCE TO A SEQUENCE LISTING

The present application includes a Sequence Listing which has been submitted in ASCII format via EFS-Web and is hereby incorporated by reference in its entirety. Said ASCII copy, created on Mar. 12, 2012, is named BHCS1126_Sequence_Listing.txt and is 1,934 bytes in size.

### BACKGROUND OF THE INVENTION

Without limiting the scope of the invention, its background is described in connection with genetic markers for recurrence prediction and determination of distant liver metastasis in primary colorectal cancers (CRCs). It will be understood by the skilled artisan that even though ˜70% of CRC metastasis is in the liver, metastasis is also possible in other organs for e.g., lung (˜20-30%), central nervous system (˜10%), adrenal glands, skeleton, spleen, skin, etc.

U.S. Patent Application Publication No. 2011/0039272 (Cowens et al. 2011) discloses a method of predicting clinical outcome in a subject diagnosed with colorectal cancer comprising determining evidence of the expression of one or more predictive RNA transcripts or their expression products in a biological sample of cancer cells obtained from the subject.

U.S. Pat. No. 7,871,769 issued to Baker et al. (2011) provides sets of genes the expression of which is important in the prognosis of cancer. In particular, the invention provides gene expression information useful for predicting whether cancer patients are likely to have a beneficial treatment response to chemotherapy FHIT; MTA1; ErbB4; FUS; BBC3; IGF1R; CD9; TP53BP1; MUC1; IGFBP5; rhoC; RALBP1; STAT3; ERK1; SGCB; DHPS; MGMT; CRIP2; ErbB3; RAP1GDS1; CCND1; PRKCD; Hepsin; AK055699; ZNF38; SEMA3F; COL1A1; BAG1; AKT1; COL1A2; Wnt.5a; PTPD1; RAB6C; GSTM1; BCL2, ESR1; or the corresponding expression product, is determined, said report includes a prediction that said subject has a decreased likelihood of response to chemotherapy.

### SUMMARY OF THE INVENTION

The present invention relates to markers for the prediction of the recurrence of distant metastasis of stage II and III primary colorectal cancer (CRC) and methods for identifying patients at high risk of metastatic recurrence, based on the presence of microsatellite alterations at selected elevated microsatellite alterations at selected tetranucleotide repeats (EMAST) and/or low levels of microsatellite instability (MSI) at mono- and dinucleotide repeat loci (MSI-L) phenotype in CRC tissues or loss of heterozygosity at the SMARCA2 region on 9p24.3.

In one embodiment, the invention provides methods for predicting probability of recurrence free survival, determining risk of recurrence, or both in a human subject suffering from primary colorectal cancer (CRC) comprising the steps of: (i) identifying the human subject suffering from the primary CRC; (ii) isolating a genomic DNA from one or more biological samples obtained from the subject, wherein the biological samples are selected from the group consisting of a frozen or fresh tissue sample; a FFPE tissue sample; a fecal sample; one or more biological fluids; or any combinations thereof; (iii) measuring or determining a level of at least one of a microsatellite instability (MSI) at a mononucleotide repeat loci, a dinucleotide repeat loci, an elevated microsatellite alteration at selected tetranucleotide repeat (EMAST) loci, or a SMARCA2R-LOH, wherein the measurement is accomplished using a microsatellite assay or microarray comprising a marker panel of at least one marker representative of each of the mono-, di- and tetranucleotide repeat loci; (iv) determining a presence or an absence of the MSI in the primary CRC from the isolated genomic DNA obtained from the human subject, wherein the determination is accomplished by amplifying the isolated genomic DNA; (v) classifying the MSI in the primary CRC into MSI-H, MSI-M and H-MSS by using a classification scheme, wherein the classification scheme comprises:
a) a high level of microsatellite instability (MSI-H) phenotype indicative of a presence of MSI at three or more of the mono- or dinucleotide markers;
b) a low level of microsatellite instability (MSI-L) phenotype indicative of a presence of MSI at least one but no more than two of the mono- or dinucleotide markers;
c) a stable level of microsatellite stability (MSS) phenotype indicative no MSI at any of the mono- or dinucleotide markers;
d) a EMAST⁺ phenotype indicative of a non MSI-H phenotype with MSI at least one of the tetranucleotide markers;
e) a EMAST⁻ phenotype indicative of a non MSI-H phenotype with no MSI at any of the tetranucleotide markers;
f) a moderate level of microsatellite instability (MSI-M) phenotype indicative of a MSI-L or EMAST⁺ or both MSI-L and EMAST⁺ phenotype; and
g) a highly stable microsatellite (H-MSS) phenotype indicative of non MSI at any of the mono-, di-, and tetranucleotide markers; and
   (vi) predicting probability of recurrence free survival, determining risk of recurrence, or both after classifying the primary CRC, wherein presence of MSI-M phenotype is indicative of a highest risk for recurrent distant metastasis, presence of MSI-H phenotype is indicative of lowest risk and H-MSS phenotype is indicative of an intermediate risk for recurrent distant metastasis in the human subject.

In specific aspects the mononucleotide repeat loci markers comprise BAT25, BAT26, or both, the dinucleotide repeat loci markers comprise D2S123; D5S346; 7S250; D18S64; 8S69; or any combinations thereof, and the tetranucleotide repeat loci markers comprise MYCL1; D20S82; D20S85; L17835; D8S321; D9S242; D19S394; or any combinations thereof. In another aspect the marker panel comprises BAT25; BAT26; D2S123; D5S346; D17S250; D18S64; D18S69; MYCL1; D20S82; D20S85; L17835; D8S321; D9S242; and D19S394. In yet another aspect a presence of the MSI-M phenotype in stage II and III primary CRC is indicative of high risk for a recurrent distant metastasis including a liver metastasis (LM) in the human subject. In another aspect wherein the method is used for treating a patient suffering from colorectal cancer; selecting anti-neoplastic agent therapy for a patient suffering from colorectal cancer; stratifying a patient in a subgroup of colorectal cancer or for a colorectal cancer therapy clinical trial; determining resistance or responsiveness to a colorectal cancer therapeutic regimen; developing a kit for diagnosis of colorectal cancer; or any combinations thereof. In one aspect, the presence of both the MSI-M and the SMARCA2R-LOH are indicative of liver metastasis from primary CRC.

Another embodiment disclosed herein relates to a method for classifying microsatellite instability (MSI) in a primary colorectal cancer (CRC) comprising: providing a panel comprising of mono-, di-, and tetranucleotide repeat loci markers to be used in a MSI assay, wherein the markers are selected from the group consisting of BAT25; BAT26; D2S123; D5S346; D17S250; D18S64; D18S69; MYCL1; D20S82; D20S85; L17835; D8S321; D9S242; and D19S394; providing a genomic DNA isolated from one or more biological samples from a human subject suffering from or suspected of suffering from the CRC; determining a presence or an absence of the MSI in the primary CRC from the isolated genomic DNA obtained from the human subject, wherein the determination is accomplished by amplifying the isolated genomic DNA; and classifying the MSI or determining a tumor phenotype based on a scheme, wherein the scheme comprises: (a) a MSI-H phenotype indicative of a presence of MSI at three or more of the mono- or dinucleotide markers; (b) a MSI-L phenotype indicative of a presence of MSI at least one but no more than two of the mono- or dinucleotide markers; (c) a MSS phenotype indicative no MSI at any of the mono- or dinucleotide markers; (d) a EMAST⁺ phenotype indicative of a non MSI-H phenotype with MSI at least one of the tetranucleotide markers; (e) a EMAST⁺ phenotype indicative of a non MSI-H phenotype with no MSI at any of the tetranucleotide markers; (f) a MSI-M phenotype indicative of a MSI-L, EMAST, or both MSI-L and EMAST phenotype; and (g) a H-MSS phenotype indicative of non MSI at any of the mono-, di-, and tetranucleotide markers. In one aspect, the method further comprises detecting the presence of a SMARCA2R-LOH, wherein the presence of both a MSI-H and SMARCA2R-LOH are indicative of liver metastasis from primary CRC.

Yet another embodiment disclosed herein relates to a biomarker for predicting probability of recurrence free survival; determining risk of recurrence; determining risk for a liver metastasis (LM); or any combinations thereof, in a human subject suffering from or suspected of suffering from primary colorectal cancer (CRC) comprising detection of a microsatellite alterations at a tetranucleotide repeat (EMAST), a low levels of dinucleotide repeat loci (MSI-L), or both in the sample, wherein a presence of a MSI-M or a MSI-M and a SMARCA2R-LOH phenotype in a majority of cells in a sample from stage II and III CRC subject is indicative of a high risk for recurrence, a high risk for liver metastasis (LM), or any combinations thereof in the human subject.

In one aspect, a determination of MSI-H, MSI-M and H-MSS are in the cells of the primary CRC is based on a panel comprising mono-, di-, and tetranucleotide repeat markers. In another aspect the panel comprises BAT25; BAT26; D2S123; D5S346; D17S250; D18S64; D18S69; MYCL1; D20S82; D20S85; L17835; D8S321; D9S242; and D19S394. In another aspect, the SMARCA2R-LOH phenotype is determined using the nucleic acids of SEQ ID NOS: 1 to 6.

The present invention also provides a kit for predicting probability of recurrence free survival, determining risk of recurrence, or both in a human subject suffering from primary colorectal cancer (CRC) comprising: biomarker detecting reagents for measuring a microsatellite instability (MSI) at a tetranucleotide repeat (EMAST), A mono- or dinucleotide repeat loci (MSI-L), or a SMARCA2R-LOH in a biological sample from a subject; and instructions for predicting probability of recurrence free survival, determining risk of recurrence, or both, wherein the instructions comprise step-by-step directions for determining presence of a MSI-M, MSI-H, H-MSS or a SMARCA2R-LOH phenotype in the biological sample obtained from a subject suffering from stage II or III CRC and comparing it with the biological obtained from a normal tissue from the same subject. In one aspect, the kit includes reagents for detecting one or more mononucleotide, dinucleotide, or tetranucleotide repeat loci markers selected from the group consisting of BAT25; BAT26; D2S123; D5S346; D17S250; D18S64; D18S69; MYCL1; D20S82; D20585; L17835; D85321; D9S242; and D19S394. In another aspect, the presence of a MSI-M phenotype or the MSI-M and SMARCA2R-LOH phenotype in a majority of cells in the sample from the subject is indicative of a high risk for recurrence and a lowered probability of recurrence-free survival in the human subject. In yet another aspect, the presence of the MSI-M phenotype in the one or more cells is indicative of a metastasis or a high risk for liver metastasis LM) in the subject. In yet another aspect the biological samples are selected from the group consisting of a frozen or fresh tissue sample, a FFPE tissue sample, a biopsy, a fecal sample, one or more biological fluids, or any combinations thereof. In one aspect, the SMARCA2R-LOH phenotype is determined using the nucleic acids of SEQ ID NOS: 1 to 6, e.g., pairs of nucleic acids therefrom.

The present invention further relates to a method for predicting probability of success of the cancer therapy, or both in a patient diagnosed with primary colorectal cancer (CRC), the method comprising: identifying the patient diagnosed with the primary CRC; and determining a level of microsatellite instability (MSI) at one or more mononucleotide, dinucleotide, tetranucleotide repeats (EMAST), or any combinations thereof in cells obtained from one or more biological samples from the patient, wherein a presence of a MSI-M, phenotype in a majority of cells in a sample from the subject is indicative of a high risk for recurrence, a high risk for distant metastasis including liver metastasis (LM), a lowered possibility of success with the cancer therapy or any combinations thereof.

One embodiment of the present invention provides a method for selecting a cancer therapy in a patient diagnosed with primary colorectal cancer (CRC), the method comprising: identifying the patient diagnosed with the primary CRC; determining a level of microsatellite instability (MSI) at one or more mononucleotide, dinucleotide, tetranucleotide repeats (EMAST), or any combinations thereof in cells obtained from one or more biological samples from the patient, wherein a presence of a MSI-M phenotype, or a MSI-M and SMARC2A-LOH phenotype in a majority of cells in a sample from the subject is indicative of a high risk for recurrence, a high risk for distant metastasis including liver metastasis (LM), a lowered possibility of success with the cancer therapy or any combinations thereof and selecting the cancer therapy based on identifying agents to lower or suppress the MSI-M. In one aspect of the method described hereinabove the step of determining the MSI further comprises the steps of: i) providing a panel comprising of mono-, di-, and tetranucleotide repeat loci markers to be used in a MSI assay, wherein the markers are selected from the group consisting of BAT25; BAT26; D2S123; D5S346; D17S250; D18S64; D18S69; MYCL1; D20S82; D20S85; L17835; D8S321; D9S242; and D19S394; ii) providing a genomic DNA isolated from one or more biological samples from the patient diagnosed with the CRC; iii) determining a presence or an absence of the MSI in the stage II and III primary CRC from the isolated genomic DNA obtained from the human subject; and iv) classifying the MSI or determining the tumor phenotype based on a scheme and categorizing CRC into 3 groups including MSI-H, MSI-M and H-MSS, wherein the scheme comprises;
(a) a MSI-H phenotype indicative of a presence of MSI at three or more of the mono- or dinucleotide markers;
(b) a MSI-L phenotype indicative of a presence of MSI at least one but no more than two of the mono- or dinucleotide markers;
(c) a MSS phenotype indicative no MSI at any of the mono- or dinucleotide markers;
(d) a EMAST phenotype indicative of a non MSI-H phenotype with MSI at least one of the tetranucleotide markers;
(e) a EMAST phenotype indicative of a non MSI-H phenotype with no MSI at any of the tetranucleotide markers;
(f) a MSI-M phenotype indicative of a MSI-L or EMAST or both MSI-L and EMAST phenotype; and
(g) a H-MSS phenotype indicative of non MSI at any of the mono-, di-, and tetranucleotide markers.

In yet another embodiment the instant invention provides a method for predicting probability of recurrence free survival, determining risk of recurrence, or both in a human subject suffering from primary colorectal cancer (CRC) comprising the steps of: i) identifying the human subject suffering from the primary CRC; ii) isolating a genomic DNA from one or more biological samples obtained from the subject, wherein the biological samples are selected from the group consisting of frozen or fresh tissue sample; a FFPE tissue sample; a fecal sample; one or more biological fluids; or any combinations thereof; iii) measuring or determining a level of a microsatellite instability (MSI) using a microsatellite assay comprising a panel of a 2 mononucleotide repeat loci, a 5 dinucleotide repeat loci, and a 7 tetranucleotide (EMAST) repeat loci selected from the group consisting of BAT25; BAT26; D2S123; D5S346; D17S250; D18S64; D18S69; MYCL1; D20S82; D20S85; L17835; D8S321; D9S242; and D19S394; iv) determining a presence or an absence of the MSI in the stage II and III primary CRC from the isolated genomic DNA obtained from the human subject, wherein the determination is accomplished by amplifying the isolated genomic DNA; v) classifying the MSI in the primary CRC by using a classification scheme, wherein the classification scheme comprises: (a) a MSI-H phenotype indicative of a presence of MSI at three or more of the mono- or dinucleotide markers, (b) a MSI-L phenotype indicative of a presence of MSI at least one but no more than two of the mono- or dinucleotide markers, (c) a MSS phenotype indicative no MSI at any of the mono- or dinucleotide markers, (d) a EMAST⁺ phenotype indicative of a non MSI-H phenotype with MSI at at least one of the tetranucleotide markers; (e) a EMAST⁻ phenotype indicative of a non MSI-H phenotype with no MSI at any of the tetranucleotide markers; (f) a MSI-M phenotype indicative of a MSI-L or EMAST or both MSI-L and EMAST phenotype; and (g) a 11-MSS phenotype indicative of non MSI at any of the mono-, di-, and tetranucleotide markers; and vi) predicting probability of recurrence free survival, determining risk of recurrence, or both after classifying the primary CRC, wherein presence of MSI-M phenotype is indicative of a highest risk for recurrent distant metastasis, presence of MSI-H phenotype is indicative of lowest risk and H-MSS phenotype is indicative of an intermediate risk for recurrent distant metastasis in the human subject.

One embodiment of the present invention discloses a method of performing a clinical trial to evaluate a candidate drug believed to be useful in treating colorectal liver metastasis, promoting recurrence-free survival, or both, the method comprising:

a) determining a level of microsatellite instability at least one of one or more tetranucleotide repeats (EMAST), a mono- and dinucleotide repeat loci (MSI-L), or a SMARCA2R-LOH, in cells obtained from a patient, wherein a MSI-M phenotype in a majority of cells in a sample from the patient is indicative of a highest risk for recurrence, a high risk for liver metastasis (LM), or any combinations thereof and presence of MSI-H phenotype is indicative of lowest risk and H-MSS phenotype is indicative of an intermediate risk for recurrent distant metastasis;
b) administering a candidate drug to a first subset of the patients, and
a placebo to a second subset of the patients;
a comparator drug to a second subset of the patients; or
a drug combination of the candidate drug and another active agent to a second subset of patients;
   c) repeating step a) after the administration of the candidate drug or the placebo, the comparator drug or the drug combination; and
   d) monitoring a recurrent-free survival rate exhibited by stage II and III primary CRC patients with an MSI-H, an MSI-M, or an H-MSS phenotype that is statistically significant as compared to the rate exhibited by the patients with the MSI-H, the MSI-M, the H-MSS and the SMARCA2R-LOH, phenotypes occurring in the second subset of patients, wherein a statistically significant increase indicates that the candidate drug is useful in treating said disease state.

In another embodiment the instant invention relates to a method for predicting probability of recurrence free survival, determining risk of recurrence, or both in a human subject suffering from stage II and III primary colorectal cancer (CRC) comprising the steps of: (i) identifying the human subject suffering from the primary CRC; (ii) isolating a genomic DNA from one or more biological samples obtained from the subject, wherein the biological samples are selected from the group consisting of a frozen or fresh tissue sample; a FFPE tissue sample; a fecal sample; one or more biological fluids; or any combinations thereof; (iii) measuring or determining a level of a microsatellite instability (MSI) using a microsatellite assay comprising a panel of a mononucleotide repeat loci, a dinucleotide repeat loci, and a tetranucleotide (EMAST) repeat loci selected from the group consisting of BAT25; BAT26; D2S123; D5S346; D17S250; D18S64; D18S69; MYCL1; D20582; D20S85; L17835; D8S321; D95242; and D19S394 or a SMARCA2R-LOH; (iv) determining a presence or an absence of the MSI in the primary CRC from the isolated genomic DNA obtained from the human subject; (v) classifying the MSI in the primary CRC by using a classification scheme and categorizing CRC into 3 groups including MSI-H, MSI-M and H-MSS, wherein the classification scheme comprises: a) a MSI-H phenotype indicative of a presence of MSI at three or more of the mono- or dinucleotide markers, b) a MSI-L phenotype indicative of a presence of MSI at least one but no more than two of the mono- or dinucleotide markers, c) a MSS phenotype indicative no MSI at any of the mono- or dinucleotide markers, d) a EMAST phenotype indicative of a non MSI-H phenotype with MSI at least one of the tetranucleotide markers, e) a EMAST phenotype indicative of a non MSI-H phenotype with no MSI at any of the tetranucleotide markers, f) a MSI-M phenotype indicative of a MSI-L or EMAST or both MSI-L and EMAST phenotype; and g) a H-MSS phenotype indicative of non MSI at any of the mono-, di-, and tetranucleotide markers; and (vi) predicting probability of recurrence free survival, determining risk of recurrence, or both after classifying the primary CRC, wherein MSI-M phenotype in a majority of cells in a sample from the patient is indicative of highest risk for recurrence, a high risk for liver metastasis (LM), or any combinations thereof and presence of MSI-H phenotype is indicative of lowest risk and H-MSS phenotype is indicative of an intermediate risk for recurrent distant metastasis.

In yet another embodiment the present invention provides a method for determining the risk for development of colorectal liver metastasis in a human subject suffering from colorectal cancer (CRC) comprising the steps of identifying the human subject suffering from the primary CRC, obtaining one or more biological samples from the subject, wherein the biological samples are selected from the group consisting of a frozen or fresh tissue sample, a FEPE tissue sample, a fecal sample, one or more biological fluids, or any combinations thereof, measuring or determining a level of a microsatellite instability (MSI) using a microsatellite assay comprising a panel of a mononucleotide repeat loci, a dinucleotide repeat loci, and a tetranucleotide (EMAST) repeat loci selected from the group consisting of BAT25; BAT26; D2S123; D5S346; D17S250; D18S64; D18S69; MYCL1; D20S82; D20S85; L17835; D8S321; D9S242; and D19S394 and a SMARCA2R-LOH, determining a presence or an absence of the MSI in the primary CRC from the isolated genomic DNA obtained from the human subject, classifying the MSI in the primary CRC by using a classification scheme, and determining the risk for colorectal cancer liver metastasis in the human subject based on a presence or an increase in the MSI-M phenotype in the sample. The classification scheme described herein comprises: i) a MSI-H phenotype indicative of a presence of MSI at three or more of the mono- or dinucleotide markers; ii) a MSI-L phenotype indicative of a presence of MSI at at least one but no more than two of the mono- or dinucleotide markers; iii) a MSS phenotype indicative no MSI at any of the mono- or dinucleotide markers; iv) a EMAST⁺ phenotype indicative of a non MSI-H phenotype with MSI at at least one of the tetranucleotide markers; v) a EMAST phenotype indicative of a non MSI-H phenotype with no MSI at any of the tetranucleotide markers; vi) a MSI-M phenotype indicative of a MSI-L or EMAST or both MSI-L and EMAST phenotype; and vii) a H-MSS phenotype indicative of non MSI at any of the mono-, di-, and tetranucleotide markers. In one aspect, the presence of both the SMARCA2R-LOH and the MSI-M are indicative of stage IV primary CRC and LM.

### BRIEF DESCRIPTION OF THE DRAWINGS

For a more complete understanding of the features and advantages of the present invention, reference is now made to the detailed description of the invention along with the accompanying figures and in which:

FIGS. 1A-1C are plots showing the Kaplan-Meier analysis for recurrence-free survival in patients with stage II and III primary CRC Recurrence-free survival rates in stage II and III CRC: FIG. 1A subdivided by MSI-H, MSI-L and MSS. MSI-H vs MSI-L (P=0.015), MSI-H vs MSS (P=0.019), MSI-L vs MSS (P=0.396), FIG. 1B subdivided by MSI-H, EMAST and non-EMAST. MSI-H vs EMAST (P=0.009), MSI-H vs non-EMAST (P=0.029), EMAST vs non-EMAST (P=0.179), and FIG. 1C subdivided by MSI-H, MSI-M and H-MSS. MSI-H vs MSI-M (P=0.008), MSI-H vs H-MSS (P=0.036), MSI-M vs H-MSS (P=0.0412) (#: not significant, *: P<0.05. P values were determined by log-rank test); and

FIGS. 2A-2E shows the MSI profile and recurrence outcome of 167 primary CRC. This figure provides detailed data from 167 primary CRCs analyzed for MSI and their outcome data as to recurrent distant metastasis. The columns depict the following: MSI data for 7 EMAST markers (MYCL1 through S321), 5 markers with CA repeats (S123 through S69), 2 markers with mono-A repeats (BAT25 and BAT26), MSI status at markers (“ ”), EMAST status, MSI-M status at NCI markers, the duration of recurrent-free survival, and the occurrence or non-occurrence of recurrent distant metastasis. For MSI data, a solid box indicates the presence of a frame-shift mutation. For MSI using the panel, L indicates MSI-L, S indicates MSS, and H indicates MSI-H. For EMAST status, E indicates EMAST-positive and non-E indicates EMAST-negative. For MSI-M status, M indicates MSI-M, HS indicates H-MSS and H indicates MSI-H. For recurrent-free survival, each number indicates number of months during which each patient was free from recurrence. For recurrence data, Y represents recurrence-positive and N represents recurrence-negative. Abbreviations used for each marker are as follows: S394: D19S394, S85: D20S85, S82: D20S82, S242: D9S242, S321: D8S321, S123: D2S123, S250: D17S250, S346: D5S346, S64: D18S64, S69: D18S69;

FIGS. 3A-3D show the MSI profile of 48 metachronous LM (FIG. 3A), 50 synchronous LM (FIG. 3B), 74 stage II and III primary CRC that gave rise to LM (FIG. 3C) and 57 stage IV primary CRC (FIG. 3D). The columns depict the following: MSI data for 7 EMAST markers (MYCL1 through S321), 5 markers with CA repeats (S123 through S69), 2 markers with mono-A repeats (BAT25 and BAT26), the MSI status at NCI markers (“NCI”), EMAST status, MSI-M status. For MSI data, a solid box indicates the presence of a frame-shift mutation. For MSI using the NCI panel, L indicates MSI-L, S indicates MSS and H indicates MSI-H. For EMAST status, E indicates EMAST-positive and non-E indicates EMAST-negative. For MSI-M status, M indicates MSI-M, HMSS indicates H-MSS and H indicates MSI-H. Abbreviations used for each marker are as follows: S394: D19S394, S85: D20S85, S82: D20S82, S242: D9S242, S321: D8S321, S123: D2S123, S250: D17S250, S346: D5S346, S64: D18S64, S69: D18S69; and

FIG. 4A shows the MSI profile of 77 LM and FIG. 4B shows the MSI profile of 77 matching primary CRC that gave rise to the LM listed in FIG. 4A. There was no change in the MSI status between these 77 matching LM and primary CRC. FIG. 4C shows the MSI profile of 9 LM and FIG. 41) shows the MSI-status of 9 matching primary CRC that gave rise to the LM listed in FIG. 4C. There was a change in MSI status between these 9 matching LM and primary CRC. The columns depict the following: MSI data for 7 EMAST markers (MYCL1 through S321), 5 markers with CA repeats (S123 through S69), 2 markers with mono-A repeats (BAT25 and BAT26). For the MSI data, a solid box indicates the presence of a frame-shift mutation. Abbreviations used for each marker are as follows: S394: D19S394, S85: D20S85, S82: D20S82: S242: D9S242, S321: D8S321, S123: D2S123, S250: D17S250, S346: D5S346, S64: D18S64, S69: D18S69.

FIGS. 5A and 5B shows the MSI-M stage II/III primary CRC and LM. FIG. 5A: The percentage of MSI-M was compared among non-metastatic stage II/III, metastatic stage II/III and stage IV cases from a Korean cohort consisting of 167 consecutive cases of primary CRC.¹⁷ FIG. 5B: The percentage of MSI-M was compared between stage II/III and stage IV that gave rise to LM and between metachronous and synchronous LM. * indicates a significant difference between 2 groups (<0.05). P values were determined using chi-square test.

FIG. 6A to 6D are MSI profile and SMARCA2R LOU in LM and primary CRC that gave rise to LM. This figure provides detailed data from FIG. 6A: 34 synchronous LM, FIG. 6B: 40 metachronous LM, FIG. 6C: 37 stage IV primary CRC, and FIG. 6D: 64 stage II/III primary CRC that gave rise to LM analyzed for MSI and LOH at SMARCA2R. The columns depict the following: mutation data for 7 EMAST markers (1 through 7), 5 markers with CA repeats (a through e), 2 markers with mono-A repeats (f and g), MSI-M status, SMARCA2R LOH status. For mutation data, a green box indicates the presence of a frame-shift mutation. For MSI-M status, M indicates MSI-M, HS indicates H-MSS and H indicates MSI-H. For LOH status, Y indicates LOU positive and N indicates LOFT negative. N.I. indicates not informative. Each number corresponds to EMAST and letter corresponds to NCI markers as follows: 1: MYCL1, 2:D19S394, 3:D20S85, 4: D20S82, 5: D9S242, 6: L17835, 7: D8S321, a: D2S123, D17S250, c: D5S346, d: D18S64, e: D18S69, f: BAT25, g: BAT26.

FIGS. 7A and 7B show that Paired LM and primary tissues whose MSI status did not change after dissemination (FIG. 7A) and the Paired LM and primary CRC tissues whose MSI status changed after dissemination (FIG. 7B).

FIG. 8A to 8C shows the SMARCA2R LOH in metastatic primary CRC and LM. FIG. 8A: The percentage of SMARCA2R-LOH is significantly higher in LM than in metastatic stage II/III primary CRC (P=0.006). FIG. 8B: The difference in percentage of SMARCA2R-LOH between metastatic stage II/III primary CRC and metachronous LM is significant (P=0.013) but not between stage IV primary CRC and synchronous LM (P=0.183). S: stage, Syn: synchronous, Meta: metachronous. FIG. 8C: A significant increase in the percentage of SMARCA2R-LOH was detected between MSI-M fraction of metastatic stage II/III primary CRC and that of metachronous LM (P=0.001). A high percentage of MSI-M positive stage IV (64%), synchronous LM (−80%) and metachronous LM (˜80%) exhibit SMARCA2R-LOH. S: stage, Syn: synchrounous, Meta: metachronous.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions which are characterized by cancer of cells of the duodenum and small intestine (jejunum and ileum).

The term “tissue sample” (the term “tissue” is used interchangeably with the term “tissue sample”) should be understood to include any material composed of one or more cells, either individual or in complex with any matrix or in association with any chemical. The definition shall include any biological or organic material and any cellular subportion, product or by-product thereof. The definition of “tissue sample” should be understood to include without limitation sperm, eggs, embryos and blood components. Also included within the definition of “tissue” for purposes of this invention are certain defined acellular structures such as dermal layers of skin that have a cellular origin but are no longer characterized as cellular. The term “stool” as used herein is a clinical term that refers to feces excreted by humans.

The term “biological fluid” as used herein refers to a fluid containing cells and compounds of biological origin, and may include blood, lymph, urine, serum, pus, saliva, seminal fluid, tears, urine, bladder washings, colon washings, sputum or fluids from the respiratory, alimentary, circulatory, or other body systems. For the purposes of the present invention the “biological fluids”, the nucleic acids containing the biomarkers may be present in a circulating cell or may be present in cell-free circulating DNA or RNA.

The term “gene” as used herein refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “allele” or “allelic form” refers to an alternative version of a gene encoding the same functional protein but containing differences in nucleotide sequence relative to another version of the same gene.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, agitated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

A “biomarker” as used herein refers to a molecular indicator that is associated with a particular pathological or physiological state. The “biomarker” as used herein is a molecular indicator for cancer, more specifically an indicator for distant metastasis of stage II and III primary CRCs. Examples of “biomarkers” include but are not limited to BAT25; BAT26; D2S123; D5S346; D11.7S250; D18S64; D18S69; MYCL1; D20S82; D20S85; L17835; D8S321; D9S242; D19S394, or combinations thereof. As used herein the term “immunohistochemistry (IHC)” also known as “immunocytochemistry (ICC)” when applied to cells refers to a tool in diagnostic pathology, wherein panels of monoclonal antibodies can be used in the differential diagnosis of undifferentiated neoplasms (e.g., to distinguish lymphomas, carcinomas, and sarcomas) to reveal markers specific for certain tumor types and other diseases, to diagnose and phenotype malignant lymphomas and to demonstrate the presence of viral antigens, oncoproteins, hormone receptors, and proliferation-associated nuclear proteins.

The term “statistically significant” differences between the groups studied, relates to condition when using the appropriate statistical analysis (e.g. Chi-square test, t-test) the probability of the groups being the same is less than 5%, e.g. p<0.05. In other words, the probability of obtaining the same results on a completely random basis is less than 5 out of 100 attempts.

The term “kit” or “testing kit” denotes combinations of reagents and adjuvants required for an analysis. Although a test kit consists in most cases of several units, one-piece analysis elements are also available, which must likewise be regarded as testing kits.

MSH3 gene (Accession No. P20585) is one of the DNA mismatch repair (MMR) genes. MSH3, together with MSH2 forms the MutSβ heteroduplex, which interacts with interstrand crosslinks (ICLs) induced by drugs such as cisplatin and psoralen. However, the precise role of MSH3 in mediating the cytotoxic effects of ICL-inducing agents remains poorly understood. The present inventors demonstrate herein the effects of MSH3 deficiency on cytotoxicity caused by cisplatin and oxaliplatin, another ICL-inducing platinum drug.

As used herein, the term “microsatellite instability” refers to a state where continuous expansion or contraction occurs in repeat units within a microsatellite sequence.

As used herein, the abbreviation EMAST refers to elevated microsatellite alterations at selected tetranucleotide repeats.

### Example 1

The present inventors show that loss of the human MutS homologue 3 (MSH3) activity results in elevated microsatellite alterations at selected tetranucleotide repeats (EMAST) and low levels of microsatellite instability (MSI) at dinucleotide repeat loci (MSI-L) in tissue cultured colon cancer cell lines (I). Microsatellite assays using markers with mononucleotide repeats alone clearly define and detect microsatellite unstable, mismatch repair (MMR)-deficient CRC with high accuracy.^{2,3} When the assay includes markers with mono- and dinucleotide repeats such as standard reference markers, a small percentage of CRC exhibiting low levels of MSI at the dinucleotide repeat markers (MSI-L) has been detected along with MSI-H, MMR-deficient CRC and microsatellite stable (MSS) CRC.³ While there are clear differences in clinicopathological behaviors or molecular profiles between MSI-H and MSI-L or between MSI-H and MSS,^{4, 5, 6, 7} the distinction between MSI-L and MSS has been long debated.^{4, 8, 9}

In colorectal cancer (CRC) tissues, 50-60% of sporadic primary tumors exhibit EMAST, and down-regulation of MSH3 is associated with MSI-L and EMAST (1). However, the pathological significance of MSI-L/EMAST and down-regulation of MSH3 in colorectal carcinogenesis is not known. Several studies have shown that the MSI-L in primary CRCs is associated with a poor prognosis. Because one of the endpoints of poor prognosis in CRC is liver metastasis (LM). When the present inventors included EMAST markers containing tetranucleotide repeats in the MSI assay in addition to the NCI markers, all of the MSI-H CRC exhibited high levels of MSI in the EMAST markers, and most but not all of the MSI-L and about a half of the MSS CRCs exhibited MSI in some of the EMAST markers.^{1,10} Furthermore, MSI-L and MSI at the EMAST loci in the sporadic CRC could be the same manifestation of loss of MSH3 protein.¹ These observations led the present inventors to hypothesize that MSI-L and/or EMAST CRCs, termed moderate levels of MSI MSI-M) in this study, may belong to a clinicopathological group that is distinctive from CRC with MSI-H and/or CRC with highly stable microsatellites (H-MSS).

The present inventors first determined the MSI status of 167 consecutive cases of primary CRC and matching normal tissues collected during the follow-up period of at least 5 years. PCR amplifications were performed from genomic DNA using 14 markers: seven standard NCI markers and seven EMAST markers. Tumors were categorized according to their MSI status using following groupings:

1) MSI-H (tumors with MSI at three or more of the seven NCI markers), MSI-L (tumors with MSI at one or two of the seven NCI markers) and MSS (tumors without MSI at any of the NCI markers);

2) MSI-H, EMAST (non-MSI-H tumors with MSI at one or more loci among seven EMAST markers), and non-EMAST (non-MSI-H tumors without MSI at any of seven EMAST markers); and

3) MSI-H, MSI-M (MSI-L and/or EMAST tumors), and H-MSS tumors without MSI at any of the 7 NCI and 7 EMAST markers.

Patients and DNA Isolation: One hundred sixty-seven consecutive cases of primary CRC and matching normal tissues were collected during the follow-up period of at least 5 years at Chonnam National University Hospital, Gwangju and Chonnam National University Hwasun Hospital, Chonnam, Republic of Korea. All of the patients received operations between 2002 and 2010. All patients provided written informed consent, and the study was approved by institutional review boards. For DNA extraction, tumor and normal tissues were micro-dissected separately from paraffin-embedded sections (10 μm). Genomic DNA was isolated and purified from micro-dissected tissues using QIAamp DNA FFPE Tissue purification kit (QIAGEN, Valencia, Calif.).

MSI Assay: To determine the MSI status of primary CRC and LM tissues, PCR amplifications were performed from genomic DNA using fluorescently labeled primers. Two markers with mononucleotide repeats (BAT25 and BAT26), five markers with dinucleotide repeats (D2S123, D5S346, D17S250, D18S64, and D18S69), and seven EMAST markers (MYCL1, D20S82, D20S85, L17835, D8S321, D9S242 and D19S394) were used. After heat denaturation, amplified PCR products were electrophoresed on an ABI PRISM 3100 Avant Genetic Analyzer (Applied Biosystems, Foster City, Calif.) and analyzed by GeneMapper fragment analysis software (Applied Biosystems). A locus was determined MSI positive when a PCR product generated from a tumor tissue exhibited at least one new peak compared to the product from a matching normal tissue.

Statistical Analysis: To estimate recurrent-free survival for a particular group of CRC, the Kaplan-Meier method was used. To evaluate a significant difference between groups, the log-rank test was used. The Cox proportional hazards regression analysis was used to evaluate the association between MSI-M and other clinicopathological factors for predicting recurrent distant metastasis. If the P value was less than 0.05, the difference was considered to be statistically significant. All statistical analysis was performed using Medcalc 7.2 (Mariakerke, Belgium).

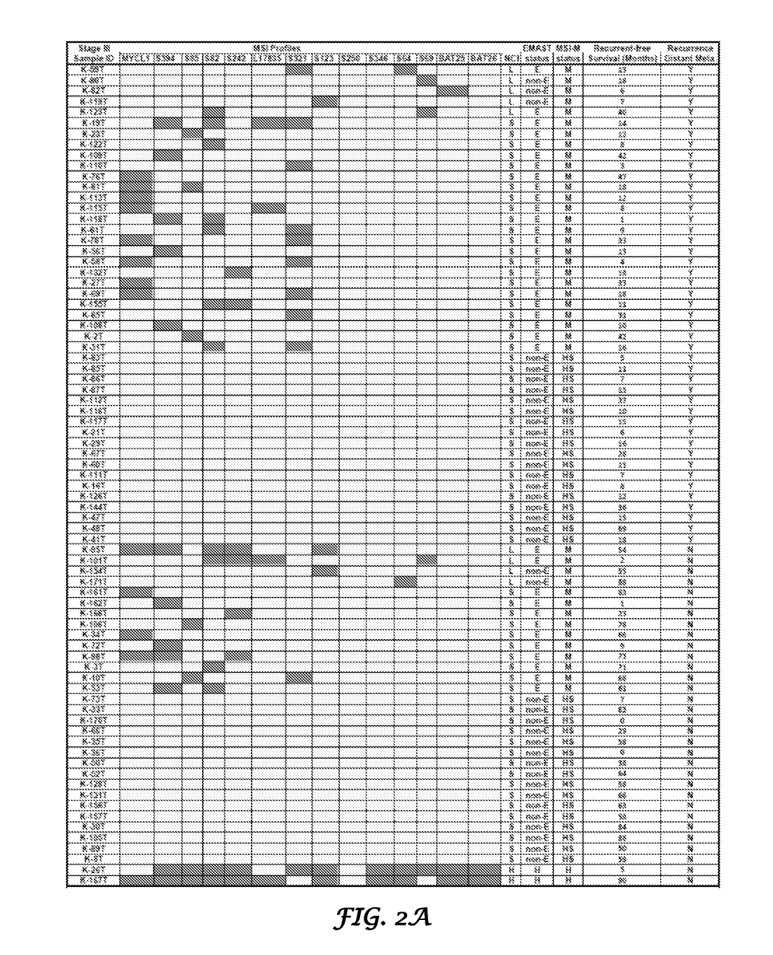

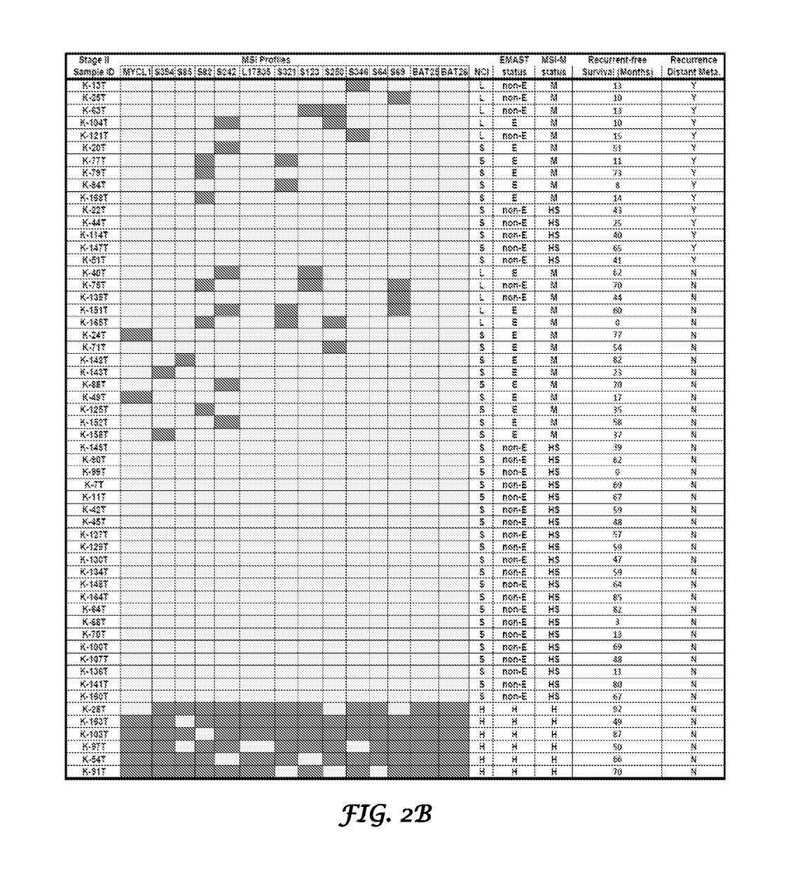

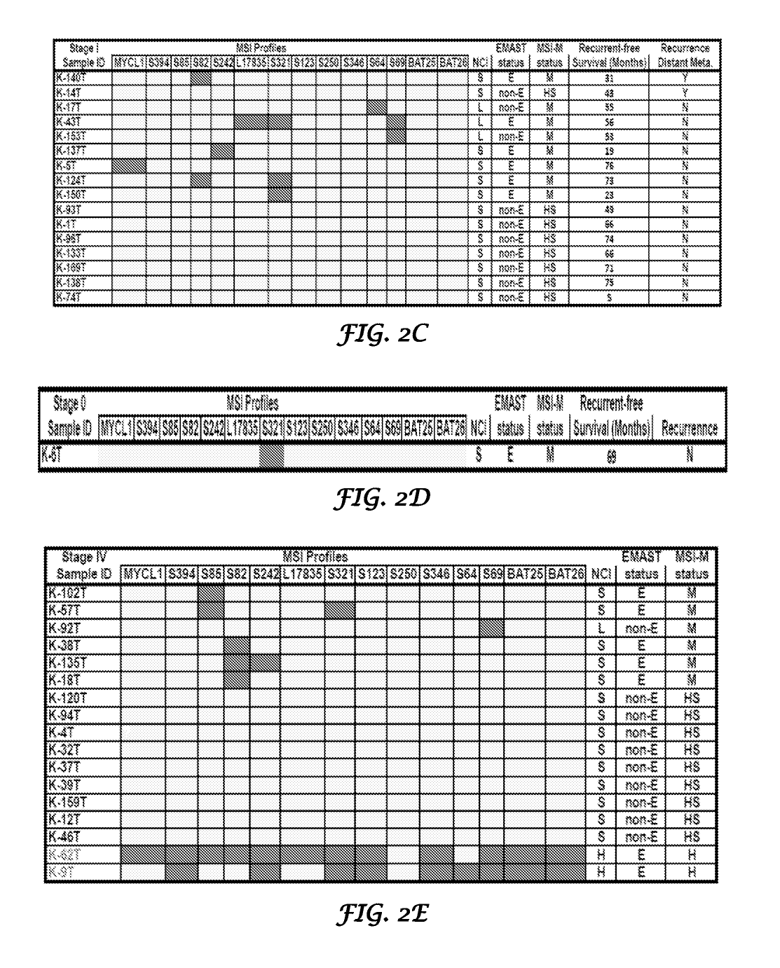

According to the definition of the present invention, 10 cases of MSI-H, 23 cases of MSI-L, 134 cases of MSS in grouping 1, 10 cases of MSI-H, 90 cases of non-EMAST, and 67 cases of EMAST in grouping 2, and 10 cases of MSI-H, 80 cases of MSI-M and 77 cases of H-MSS in grouping 3 were identified (FIGS. 2A-2E). No significant association was found between MSI-M (Table 2) or other categories of CRC (not shown) and clinicopathological characteristics such as age, sex, tumor grade, location, stage and presence or absence of adjuvant chemotherapy.

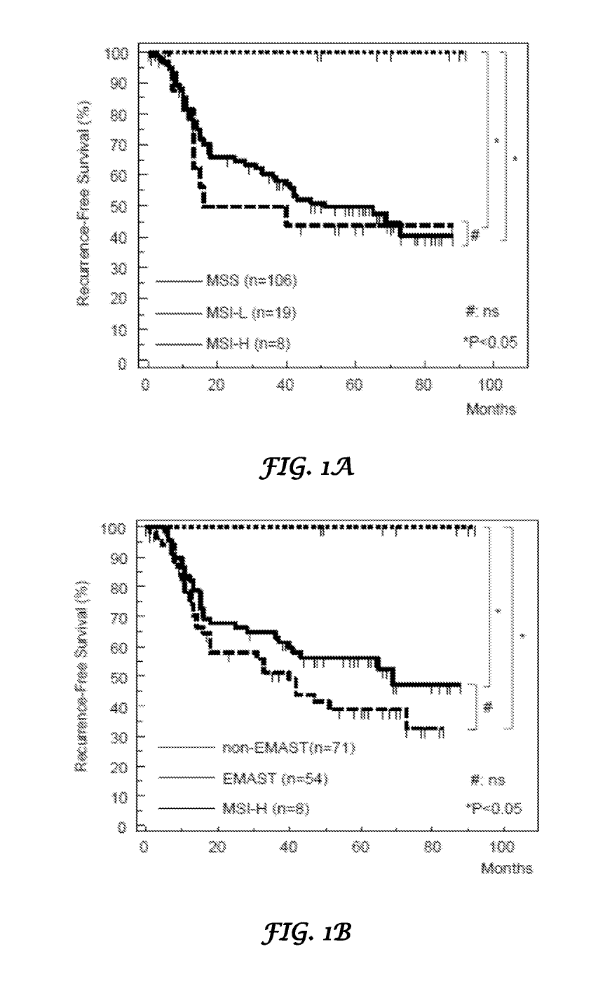

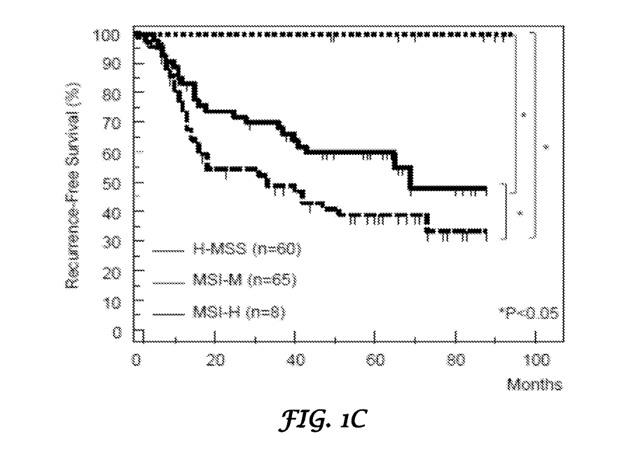

When the inventors estimated the recurrence-free survival of 133 cases of stage II and III primary CRC using the Kaplan-Meier method, there was a significant difference in recurrence-free survival between MSI-H and MSI-L (FIG. 1A, P=0.015) or between MSI-H and MSS (FIG. 1A, P=0.019) but no difference in recurrence-free survival between MSI-L and MSS (P=0.396) in grouping 1. Similarly, a significant difference was detected between MSI-H and EMAST FIG. 1B, P=0.009) and between MSI-H and non-EMAST (FIG. 1B, P=0.029) but not between EMAST and non-EMAST (FIG. 1B, P=0.179) in grouping 2. In contrast, the MSI-H, MSI-M and H-MSS patients in grouping 3 showed significantly different rates of recurrence-free survival from each other (FIG. 1C). MSI-M tumors were more likely to recur as distant metastasis than were H-MSS (FIG. 1C, P=0.0415). Only when MSI-L and EMAST were put into the same group as MSI-M could they be recognized as a high-risk group among the non-MSI-H patients. Furthermore, when compared to H-MSS by multivariate Cox proportional hazard analysis, MSI-M is an independent predictor for recurrent distant metastasis from stage II and III primary CRC (Table I, Hazard Ratio: 1.83, 95% CI:1.06-3.15, P=0.03). The results reported herein indicate that MSI-M is a predictable marker for recurrent distant metastasis of stage II and III primary CRC and can be used for identifying high-risk patients.

TABLE 1
Multivariate analysis for recurrent distant metastasis
of stage II and III primary CRC.

| Factors | Hazard Ratio | 95% CI | P values |
|---|---|---|---|
| MSI-M vs H-MSS | 1.83 | 1.06-3.15 | 0.03 |
| Age: ≦62 vs >62 | 0.91 | 0.52-1.56 | 0.73 |
| Male vs female | 1.04 | 0.60-1.80 | 0.87 |
| Grade^{a}: G2 + G3 vs G1 | 1.77 | 1.00-3.12 | 0.051 |
| Location^{b}: distal vs proximal | 1.47 | 0.64-3.35 | 0.35 |
| Chemotherapy^{c}: yes vs no | 1.7 | 0.60-4.76 | 0.31 |
| Stage: III vs II | 2.17 | 1.16-4.05 | 0.015 |

TABLE 2
Relationship between MSI-M and clinicopathological
characteristics of primary CRC.

| | | No. of patients | |
|---|---|---|---|
| | No. of patients | with MSI-M (%) | P values |
| Age | | | |
| ≦62 | 79 | 40 (50.6) | |
| >62 | 88 | 40 (45.5) | 0.504 |
| Sex | | | |
| Female | 70 | 28 (40.0) | |
| Male | 97 | 52 (53.6) | 0.082 |
| Grade^{a} | | | |
| G1 | 67 | 36 (53.7) | |
| G2 plus G3 | 100 | 44 (44.0) | 0.217 |
| Location^{b} | | | |
| Proximal | 30 | 13 (43.3) | |
| Distal | 137 | 67 (48.9) | 0.58 |
| Chemo^{c} | | | |
| Yes | 119 | 56 (47.1) | |
| No | 31 | 17 (54.8) | 0.44 |
| Stage | | | |
| I-II | 72 | 33 (45.8) | |
| III-IV | 95 | 47 (49.5) | 0.641 |

^{a}G1: well differentiated, G2: moderately differentiated, G3: poorly differentiated.
^{b}Proximal includes cecum, ascending and traverse colon. Distal includes sigmoid colon and rectal.
^{c}Some patients (stage II and III) received 5-FU-based adjuvant chemotherapy. Others did not.
NOTE.
All P values were calculated by the chi-square test.
^{a}G1: well differentiated, G2: moderately differentiated, G3: poorly differentiated.
^{b}Proximal includes cecum, ascending and traverse colon. Distal includes sigmoid colon and rectal.
^{c}Some patients (stage I, II and III) received 5-FU-based adjuvant chemotherapy. Others did not.

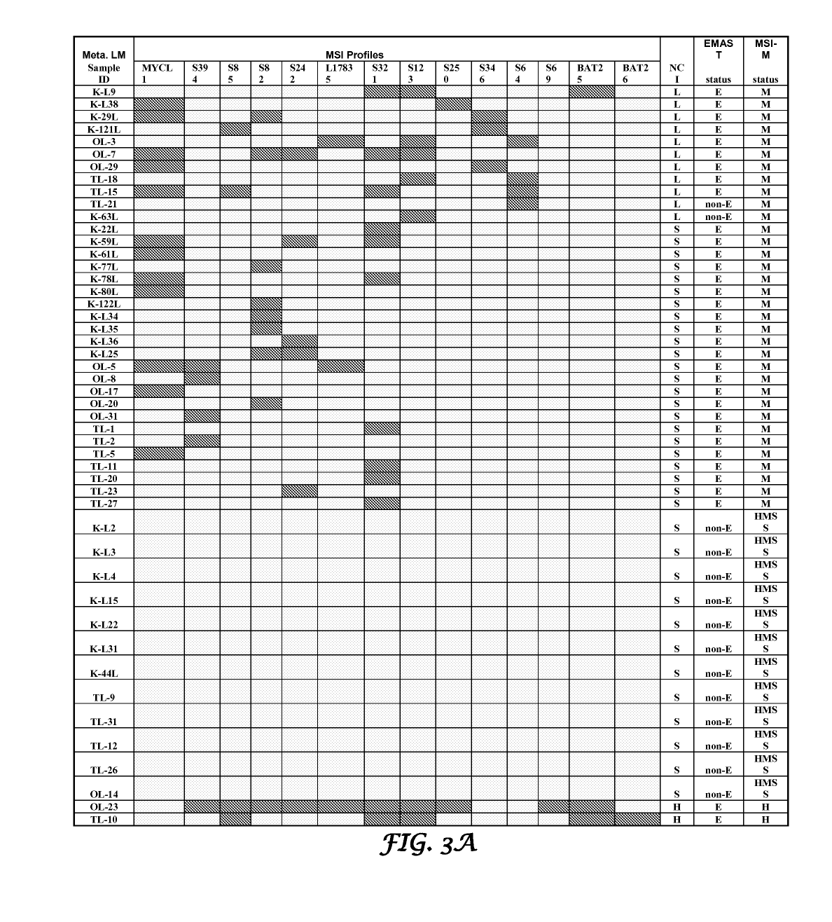

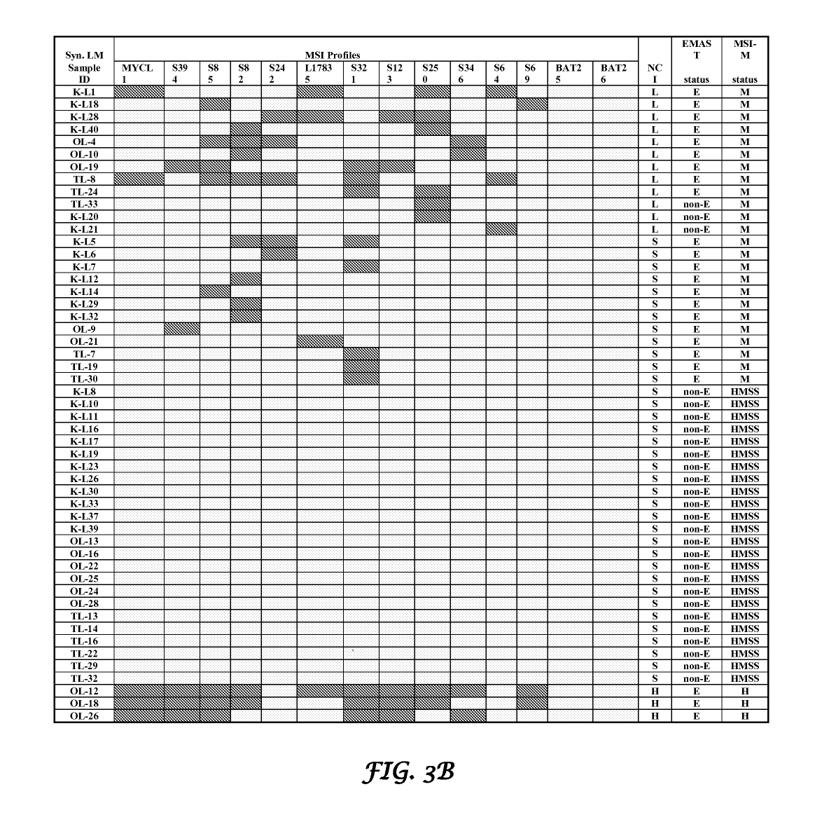

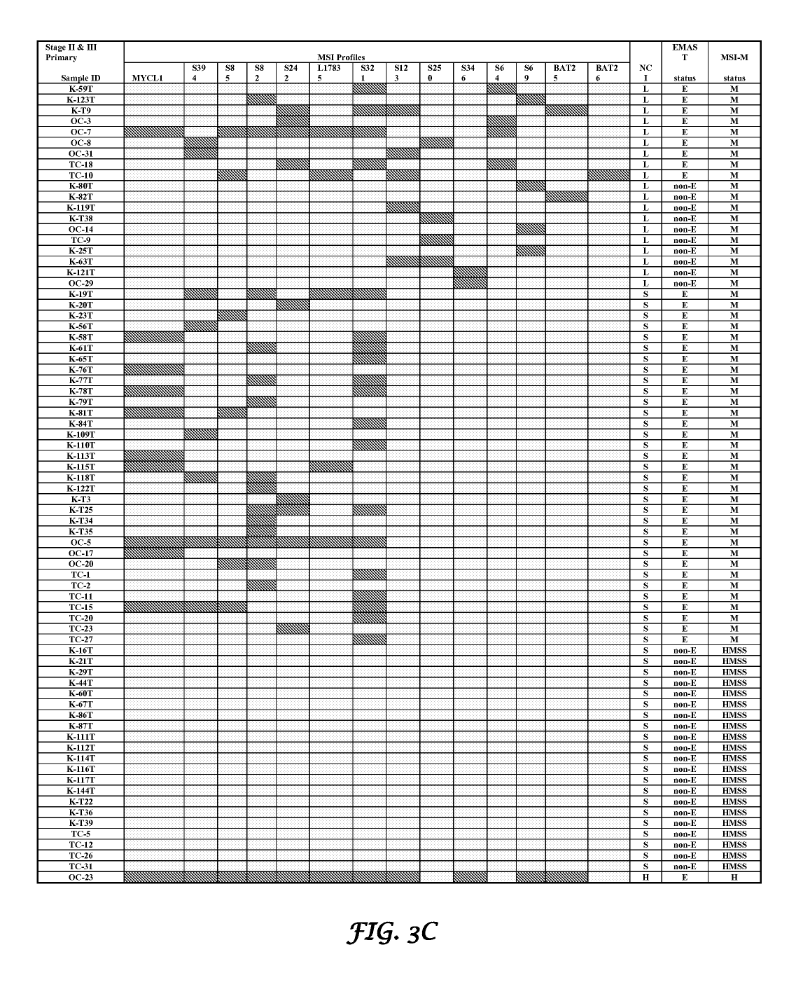

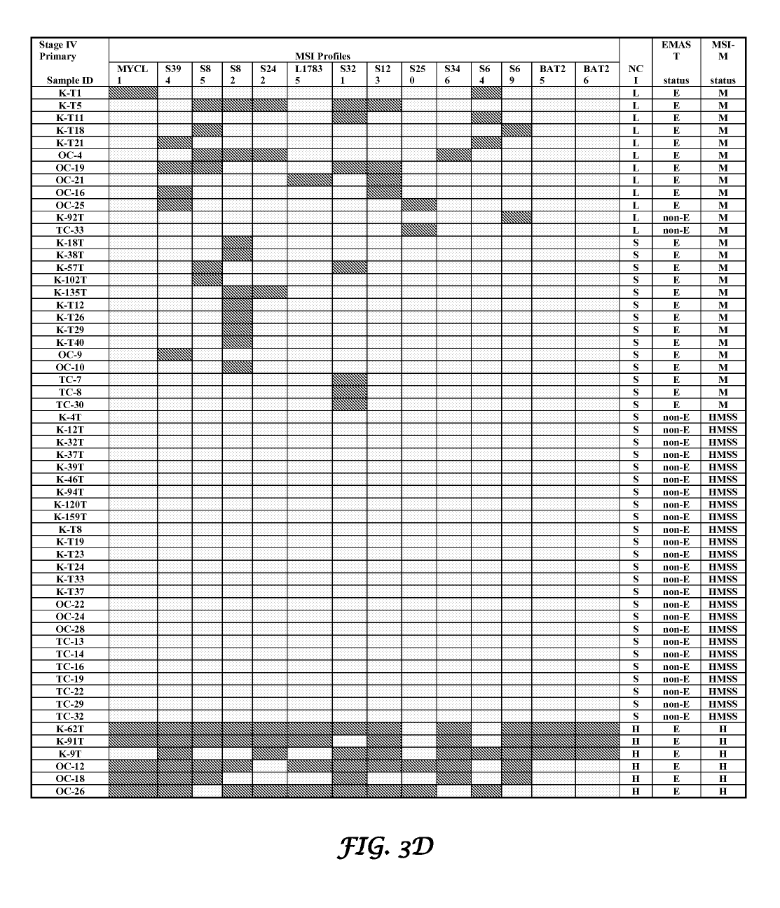

The present inventors have now recognized that MSI-M in stage II and III primary CRCs may be associated with ability to metastasize to the liver (metachronous liver metastasis). The inventors analyzed-the MSI status of 98 liver metastasic (LM) tissues (48 metachronous and 50 synchronous) (FIG. 3A, and FIG. 3B) and 131 metastatic primary CRC tissues that gave rise to LM (56 stage III and 18 stage II and 57 stage IV,) (FIG. 3C, and FIG. 3D). FIGS. 3A-3D show the MSI profile of 48 metachronous LM (FIG. 3A), 50 synchronous LM (FIG. 3B), 74 stage II and III primary CRC that gave rise to LM (FIG. 3C) and 57 stage IV primary CRC (FIG. 3D). The columns depict the following: MSI data for 7 EMAST markers (MYCL1 through S321), 5 markers with CA repeats (S123 through S69), 2 markers with mono-A repeats (BAT25 and BAT26), the MSI status at NCI markers (“NCI”), EMAST status, MSI-M status. For MSI data, a solid box indicates the presence of a frame-shift mutation. For MSI using the NCI panel, L indicates MSI-L, S indicates MSS and H indicates MSI-H. For EMAST status, E indicates EMAST-positive and non-E indicates EMAST-negative. For MSI-M status, NI indicates MSI-M, HMSS indicates H-MSS and H indicates MSI-H. Abbreviations used for each marker are as follows: S394: D19S394, S85: D20S85, S82: D20S82, 5242: D9S242, 5321: D8S321, S123: D2S123, S250: D175250, S346: D5S346, S64: D18S64, S69: D18S69.

Among 48 metachronous LM, 70.8% (34/48 cases) showed MSI-M (FIG. 3A, Table 3). In contrast to metachronous LM, 46.0% of synchronous LM (23 of 50 cases) showed MSI-M (FIG. 3B). The difference in the frequency of MSI-M between synchronous and metachronous LM is significant (Table 3, p=0.013). When the inventors performed multivariable logistic regression analysis to compare the factors associated with metachronous and synchronous LM (Table 3), the results confirmed that MSI-M is significantly associated with metachronous LM compared to synchronous LM (Odds ratio: 3.54, 95% CI: 1.41-8.93. P=0.007), and further showed that primary CRCs from which metachronous LMs originated are associated with well-differentiated state (P=0.02) and with distal location (P=0.01). (Table 3).

The inventors next examined the MSI status of 1130 metastatic primary CRC that gave rise to LM. Among them, 74 cases were stages II or III and 56 cases were stage IV and (FIGS. 3C and 3D). 70.3% of the stage II and III primary CRC that gave rise to LM (52/74 cases) were positive for MSI-M (FIG. 3C) whereas 48.2% of stage IV CRC (27/56 cases) exhibited MSI-M (FIG. 3D), and this difference was significant (P=0.012) (Table 4). A significantly higher frequency of MSI-M was observed in the stage II and III primary CRC that gave rise to LM (P=0.007) than the average frequency of MSI-M in stage II and III primary CRC (48.4%). In contrast, a frequency of MSI-M was similar between the stage IV primary CRC and total stage II and III primary CRC (48.2% versus 48.4%). Multivariable logistic regression analysis also confirmed that MSI-M is associated with stage II and III primary CRC that gave rise to metachronous LM compared to stage IV primary CRC that gave rise to synchronous LM (Odds ratio: 2.61, 95% CI: 1.218-5.591, P=0.0137, Table 4).

TABLE 3
MSI-M is enriched in metachronous LM compared to synchronous LM.

| | Univariate Analysis^{a} | | | | | | |
|---|---|---|---|---|---|---|---|
| | No. of | | No. of | | | Multivariate | |
| | metachronous | | synchronous | | | Analysis^{b} | |
| Factors | LM (%) | | LM (%) | | P values | OR | P values |
| Age | | | | | | | |
| ≦62 | 26 | (54.1) | 22 | (44.0) | | | |
| >62 | 22 | (45.9) | 28 | (56.0) | 0.314 | 0.73 | 0.494 |
| Sex | | | | | | | |
| F | 17 | (35.4) | 19 | (38.0) | | | |
| M | 31 | (64.6) | 31 | (62.0) | 0.791 | 1.12 | 0.809 |
| Grade^{c} | | | | | | | |
| G1 | 18 | (37.5) | 8 | (16.0) | | | |
| G2 + G3 | 30 | (62.5) | 42 | (84.0) | 0.016 | 0.26 | 0.012 |
| Location^{d} | | | | | | | |
| Proximal | 3 | (6.3) | 12 | (24.0) | | | |
| Distal | 45 | (93.7) | 38 | (76.0) | 0.015 | 6.32 | 0.014 |
| MSI | | | | | | | |
| non-MSI-M | 14 | (29.2) | 27 | (54.0) | | | |
| MSI-M | 34 | (70.8) | 23 | (46.0) | 0.013 | 3.54 | 0.007 |
| Population | | | | | | | |
| Japanese | 25 | (52.1) | 26 | (52.0) | | | |
| Korean | 23 | (47.9) | 24 | (48.0) | 0.993 | 0.75 | 0.541 |
| Total | 48 | | 50 | | | | |

^{a}P values were determined by chi square test.
^{b}Multivariate logistic-regression analysis were performed to determine the factors associated with metachronous LM
^{c}A degree of differentiation exhibited by primary CRCs from which the LMs originated. G1: well differentiated, G2: moderately differentiated, G3: poorly differentiated.
^{d}A location of primary CRCs from which the LMs originated. Proximal includes cecum ascending and traverse colon. Distal includes sigmoid colon and rectal.

TABLE 4
MSI-M is enriched in primary II and III that gave rise to LM.

| | Univariate Analysis^{a} | | | | | Multivariate | |
|---|---|---|---|---|---|---|---|
| | No. of | | No. of | | | Analysis^{b} | |
| | primary II and III | | primary IV | | P | | P |
| Factors | (%) | | (%) | | values | OR | values |
| Age | | | | | | | |
| ≦62 | 41 | (55.4) | 29 | (51.8) | | | |
| >62 | 33 | (44.6) | 27 | (48.2) | 0.416 | 1.05 | 0.9039 |
| Sex | | | | | | | |
| F | 26 | (35.1) | 25 | (44.6) | | | |
| M | 48 | (64.9) | 31 | (55.4) | 0.272 | 1.46 | 0.328 |
| Grade^{c} | | | | | | | |
| G1 | 24 | (32.4) | 12 | (21.4) | | | |
| G2 + G3 | 50 | (67.6) | 44 | (78.6) | 0.165 | 0.58 | 0.2161 |
| Location^{d} | | | | | | | |
| Proximal | 8 | (10.8) | 16 | (28.6) | | | |
| Distal | 66 | (89.2) | 40 | (71.4) | 0.01 | 2.64 | 0.0537 |
| MSI | | | | | | | |
| non-MSI-M | 22 | (29.7) | 29 | (51.8) | | | |
| MSI-M | 52 | (70.3) | 27 | (48.2) | 0.012 | 2.61 | 0.0137 |
| Population | | | | | | | |
| Japanese | 22 | (29.70 | 24 | (42.9) | | | |
| Korean | 52 | (70.3) | 32 | (57.1) | 0.121 | 1.79 | 0.1471 |
| Total | 74 | | 56 | | | | |

^{a}P values were determined by chi square test.
^{b}Multivariate logistic-regression analysis were performed to determine the factors associated with metachronous LM
^{c}A degree of differentiation exhibited by primary CRCs from which the LMs originated. G1: well differentiated, G2: moderately differentiated, G3: poorly differentiated.
^{d}A location of primary CRCs from which the LMs originated. Proximal includes cecum ascending and traverse colon. Distal includes sigmoid colon and rectal.

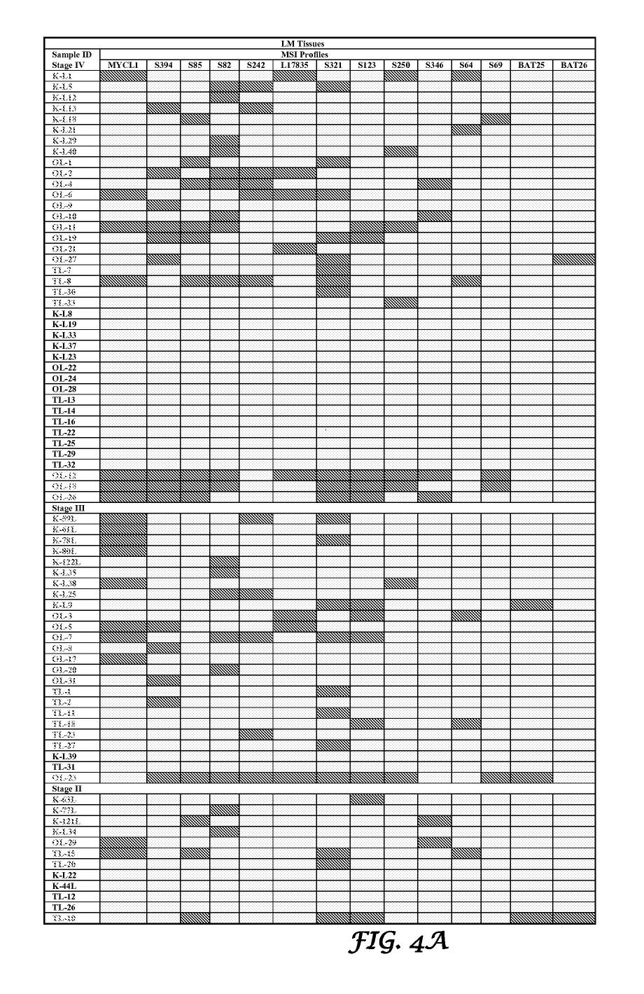

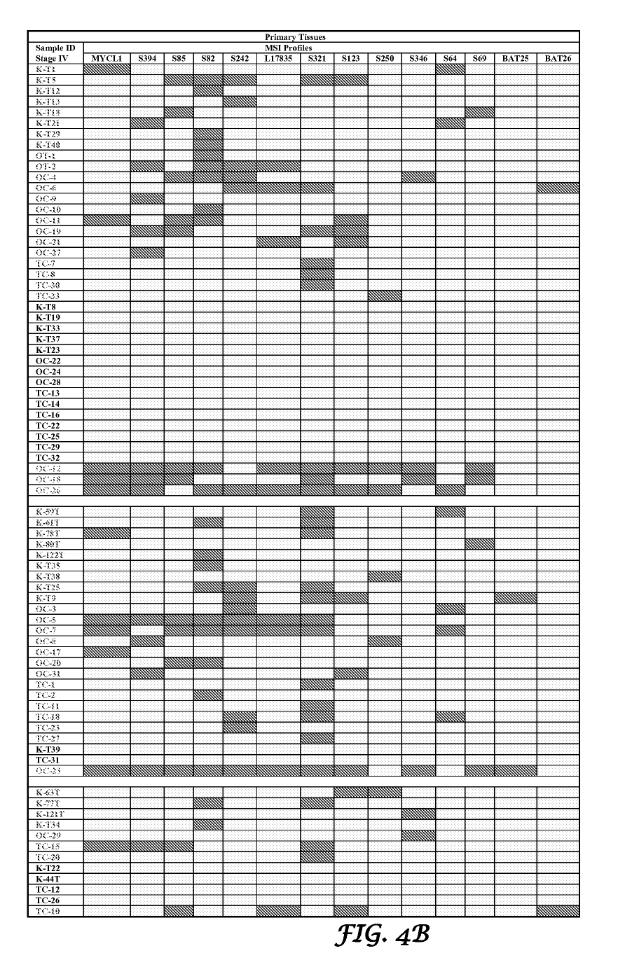

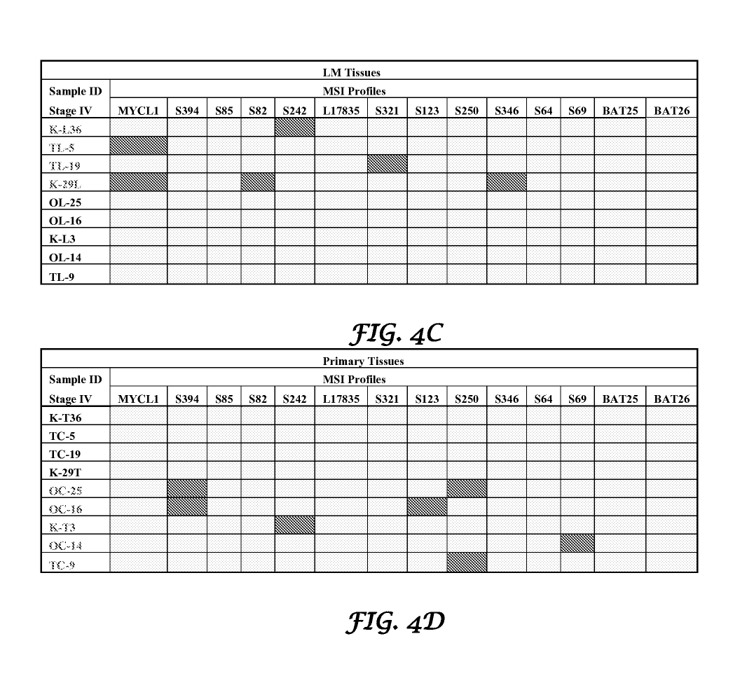

To determine whether the MSI profile changes after dissemination, the present inventors compared the MSI status of 86 matched LMs (FIG. 4A) and primary CRCs from which these LMs originated (FIG. 4B). It was found that the MSI status changed only in 9 matched cases (10.5%), including 4 cases where the MSI status changed from MSS to MSI-M and 5 cases where the MSI status changed from MSI-M to MSS after dissemination (FIG. 4C). These results indicate that the MSI status of primary CRC reflects those of metastasized tissues in most of the cases (90%) (FIG. 4D).

FIG. 4A shows the MSI profile of 77 LM and FIG. 4B shows the MSI profile of 77 matching primary CRC that gave rise to the LM listed in FIG. 4A. There was no change in the MSI status between these 77 matching LM and primary CRC. FIG. 4C shows the MSI profile of 9 LM and FIG. 4D shows the MSI-status of 9 matching primary CRC that gave rise to the LM listed in FIG. 4C. There was a change in MSI status between these 9 matching LM and primary CRC. The columns depict the following: MSI data for 7 EMAST markers (MYCL1 through S321), 5 markers with CA repeats (S123 through S69), 2 markers with mono-A repeats (BAT25 and BAT26). For the MSI data, a solid box indicates the presence of a frame-shift mutation. Abbreviations used for each marker are as follows: S394: D19S394, S85: D20S85, S82: D20S82, S242: D9S242, 5321: D8S321, S123: D2S123, S250: D17S250, S346: D5S346, S64: D18S64, 569: D18S69.

The findings of the present invention indicate that stage II and III patients with MSI-M, had a shorter recurrence-free survival than the rest of patients with high levels of MSI (MSI-H) (P=0.0084) or with highly stable microsatellites (P=0.0415) by Kaplan-Meier analysis, and that MSI-M is an independent predictor for recurrent distant metastasis in primary stage II and III CRCs regardless absence or presence of adjuvant chemotherapy (Cox proportional hazard analysis, Risk Ratio: 1.83, 95% CI: 1.06-3.15, P=0.0301). Furthermore, studies conducted by the present inventors indicate that MSI-M in primary CRCs may be associated with ability to form metachronous metastasis to the liver. The findings presented herein suggest that the biology of metachronous LMs from stage II and III might be different from those synchronous LMs which came from cases that were stage IV at initial staging, leading to the hypothesis that the MSI-M pathway plays a more prominent role in the metachronous liver metastatic than synchronous liver metastasis.

### Example 2

### SMARCA2R LOH and MSI-M in Liver Metastasis from CRC

Example 1 demonstrated that moderate microsatellite instability (MSI-M) defined by NCI reference markers and elevated microsatellite alterations at selected tetranucleotide repeats (EMAST) markers was common in primary CRC, and was an independent predictor for recurrent distant metastasis of stage II and III (II/III) primary CRC. However, how MSI-M is linked to recurrent distant metastasis is not known. To identify genetic changes or markers significantly associated with MSI-M and with liver metastasis (LM) from primary CRC, 57 pairs of matching metastatic primary CRC and corresponding liver metastasis (LM) from the same patients and 17 cases of LM for microsatellite instability (MSI) using 7 NCI reference markers and 7 EMAST markers. Association of MSI-M with clinicopathological factors was determined using the chi-square test. A total of 142 gene loci were selected with polymorphic microsatellites by genome data mining, and examined each locus for MSI and loss of heterozygosity (LOH) in 24 LM exhibiting MSI-M. Because LOH at SMARCA2 on 9p24.3 was frequently found in MSI-m-positive LM (64%), we further examined LOH status at the SMARCA2 region (SMARCA2R-LOH) in an additional 50 cases of LM and 224 cases of primary CRC. Association of SMARCA2R-LOH with MSI-M, LM or other clinicopathological factors was determined using the chi-square test.

Abbreviations: colorectal cancer (CRC), liver metastasis (LM), microsatellite instability (MSI), elevated microsatellite alterations at selected tetranucleotide repeats (EMAST), loss of heterozygosity (LOH), low levels of MSI (MSI-L), high levels of MSI (MSI-H), moderate MSI (MSI-M), LOH at the SMARCA2 region (SMARCA2R-LOH).

The frequency of MSI-M in metastatic stage II/III primary CRC was significantly higher than that of MSI-M in non-metastatic stage II/III primary CRC or in stage IV primary CRC. MSI status did not change between LM and the primary CRC from which the LM derived. Thus, MSI-M was more significantly frequent in metachronous LM than in synchronous LM. The frequency of SMARCA2R-LOH in metachronous LM was significantly higher than that of metastatic stage primary CRC from which the metachronous LM originated, suggesting that SMARCA2R-LOH may contribute to the metastasis process after dissemination. Furthermore, this increase was restricted in MSI-M population of metachronous LM. Thus, while there was no association between MSI-M and SMARCA2R-LOH in stage II/III primary CRC that gave rise to LM, there was a significant association between them in metachronous LM. In contrast, while there was no difference in the frequency of SMARCA2R-LOH in synchronous LM compared to that found in stage IV primary CRC, a significant association between MSI-M and SMARCA2R-LOH was detected in stage IV primary CRC and synchronous LM. Thus, MSI-M and SMARCA2R-LOH coexisted in a large fraction (70-80%) of stage IV primary CRC, metachronous LM or synchronous LM tissues.

Microsatellite instability (MSI) is a state where continuous expansion or contraction occurs in repeat units within a microsatellite sequence. Defects in mismatch repair (MMR) systems fail to repair slippage errors generated by DNA polymerase in microsatellite loci, resulting in MSI.¹ Tumor tissues derived from MMR-defective cases generally exhibit a high level of MSI (MSI-H).²

Although different markers can be used to identify CRC with defective MMR, an assay using markers with only mononucleotide repeats clearly defines and detects this type of CRC with high accuracy.^{3, 4} When markers with mono- or dinucleotide repeats, such as NCI reference markers, were used, CRC with low MSI (MSI-L) at dinucleotide repeat was detected along with MS and microsatellite stable (MSS) CRCs ². Most of the MSI-L sporadic CRC have acquired a silenced hMLH1 by promoter hypermethylation ⁵ and have a better prognosis than MSI-L and/or MSS CRC, ⁶⁻⁸ Thus, the distinction between MSI-H and MSI-L/MSS CRC is genetically and phenotypically clear. In contrast, although MSI-L CRC does not have a defect in hMSH2 or hMLH1,² the molecular basis of MSI-L has been largely unknown. Furthermore, MSI-L and MSS CRC have similar clinicopathological phenotypes in some studies.^{2, 9} These Observations suggest that most CRC may exhibit some level of MSI if enough markers are examined and that MSI-L may be no different than MSS CRC. ^{2, 9, 10} However, several studies have shown that MSI-L is different from MSS CRC¹¹⁻¹³. Gene expression profiles among MSI-H, MSI-L and MSS CRC are different from each other and each CRC type exhibits a distinct set of gene expressions. ¹¹ Two independent studies have demonstrated that Duke C MSI-L CRC has a poor prognosis, probably due to its association with recurrence^{12, 13}. In addition to MSI defined by NCI markers, another type of mutation in microsatellite loci has been Observed in human cancers.^{14, 15} Among non-MSI-H CRC, some tumors show instability at loci with tetranucleotide repeats containing aaag or agat ¹⁶⁻¹⁸ but not at loci with mononucleotide repeats¹⁶. This type of microsatellite alteration is called EMAST. Although the association between mutations in p53 and EMAST has been demonstrated in non-small cell lung cancers, ¹⁹ the clinicopathological significance and molecular basis of EMAST in CRC has not been well understood¹⁷.

Hereinabove the present inventors demonstrated that MSI-L and EMAST may both be a consequence of MSH3-deficiency and may belong to the same pathological group of CRCs. ²⁰ About 50% of non-MSI-H primary CRC exhibited EMAST when 7 EMAST loci were examined for MSI. ^{16, 17} Most but not all MSI-L and half the MSS defined by standard NCI markers exhibited EMAST. ^{16, 17} Loss of MSH3 in tissue cultured colon cancer cells resulted in MSI at EMAST loci and low MSI at loci with dinucleotide repeats. ¹⁶ A significant association between down-regulation of MSH3 expression and MSI-L/EMAST was detected in CRC tissues. ¹⁶ Finally, when a cohort of 167 primary CRC was examined for MSI using 7 standard NCI markers and 7 EMAST markers, three independent groups of stage II and III CRC that differ according to the risk of recurrent distant metastasis were recognized. ²⁰ The highest risk group exhibited MSI-L and/or EMAST. The lowest risk group exhibited MSI-H, and the intermediate risk group showed highly stable microsatellite MSS). Based on these findings, we proposed to define MSI-L/EMAST as one group and named this group of CRC moderate MSI (MSI-M).²⁰ However, it remained to be determined how MSI-M is linked to recurrence and/or distant metastasis in CRC.

In this study, evidence was developed that MSI-M is involved in liver metastasis (LM) from primary CRC. We identify the genetic changes associated with MSI-M in LM tissues, 142 candidate genes were selected with intragenic microsatellites by genome data mining and screened them for a high frequency of MSI and LOH in 24 LM tissues that exhibited MSI-M. The present inventors determined that 1) LM tissues should contain all genetic and/or epigenetic changes necessary for metastasis to the liver, 2) a gene containing microsatellite with di-, tri- or tetra-nucleotide repeats can be a target of a mechanism that induces MSI-M. Such a gene may be enriched in MSI-M-positive LM, 3) because the studies in Example 1 showed that EMAST (MSI-M) is associated with frequent LOH events at certain gene loci,¹⁷ LOH at the specific gene locus could be selected along with MSI-M. Some of these loci may play a role for LM formation. Among the gene loci exhibiting a high frequency of LOH or MSI in MSI-M-positive LM, SMARCA2R-LOH on 9p24.3 was associated with MSI-M in LM and stage IV primary CRC tissues but not in stage II and III primary CRC tissues. This example shows that two events, one associated with MSI-M and another with SMARCA2R-LOH, leads cancer cells to become competent for metastasis to the liver.

Materials and Methods. Tissues and DNA isolation. 167 consecutive cases of primary CRC and matching normal tissues were collected during a follow-up period of at least 5 years at Chonnam National University Hospital, Gwangju and Chonnam National University Hwasun Hospital, Chonnam, Republic of Korea.²⁰ Thirty-one pairs of matching metastatic primary CRC tissues and corresponding liver metastasis (LM) tissues from the same patients and 17 cases of LM tissues were collected from the archives of the Department of Pathology at Chonnam National University. All of the cases received operations between 2002 and 2010. We also obtained 26 pairs of matching sporadic metastatic primary CRC tissues and corresponding LM tissues collected at Toho University, Ohmori Hospital (Tokyo, Japan). All patients provided written informed consent, and studies were approved by institutional review boards. For DNA extraction, tumor and normal tissues were micro-dissected separately from paraffin-embedded sections (10 μm). Genomic DNA was isolated and purified from micro-dissected tissues using a QIAamp DNA FFPE Tissue purification kit (QIAGEN, Valencia, Calif.).

MSI and LOH Analysis. To determine the MSI status of primary CRC and LM tissues, PCR amplifications were performed from genomic DNA using fluorescently labeled primers. Two markers with mononucleotide repeats (BAT25 and BAT26), five markers with dinucleotide repeats (D2S123, D5S346, D17S250, D18S64, and D18S69), and seven EMAST markers (MYCL1, D20S82, D20S85, L17835, D8S321, D9S242 and D19S394) were used. ¹⁷ Tumors were categorized as: 1) a high level of MSI (MSI-H): tumors exhibiting MSI at three or more of the seven mono- or dinucleotide markers; 2) a moderate level of MSI (MSI-M): tumors exhibiting MSI at one or two of the seven mono-, and dinucleotide markers (MSI-L) and/or tumors exhibiting MSI at one or more than one locus among the seven EMAST markers (EMAST); 3) highly stable microsatellites (H-MSS): tumors which did not exhibit MSI at any of the 14 markers.

For 142 gene loci (see below) containing polymorphic di-, tri- or tetranucleotide repeats, the genomic sequences from both 5′ and 3′ ends of the repeats were used to design PCR primers by online software Prim3Plus (http://www.bioinformatics.nl/cgi-bin/primer3plus/primer3plus.cgi). Amplification of these loci and detection of MSI or LOH were performed by the method described by Schuelke. ²² After heat denaturation, amplified PCR products were electrophoresed on an ABI PRISM 3100 Avant Genetic Analyzer (Applied Biosystems, Foster City, Calif.) and analyzed by GeneMapper fragment analysis software (Applied Biosystems).

A locus was determined MSI positive when a PCR product generated from tumor tissue exhibited at least one new peak compared to the product from matching normal tissue. When a normal tissue exhibited heterozygosity at a particular marker, LOH was assessed in the corresponding tumor tissue. The height of the electrophoregram of PCR product was used as a measure for signal intensity. The ratio of signal intensities between two alleles in normal cells and the ratio of signal intensities between two alleles in the corresponding tumor cells were compared. When the ratio in tumor cells exhibited less than 45% of the ratio in normal cells, the locus was determined to be LOH positive.

Screening for a gene associated with MSI-M and LM. In total, we selected 142 genes with di-, tri- or tetranucleotide repeats for screening. The main criteria for selection of these genes were: 1) microsatellite repeats were at 5′-UTR, exon, 3′UTR or intron of a gene, 2) the repeats were large enough to be susceptible for DNA polymerase error, and 3) the repeats were polymorphic in length so that LOH could be detected. An NCBI blast search (blast.ncbi.nlm.nih.gov/Blast.cgi) followed by accessing the Ensemble Database (www.ensembl.org/index.html) to detect polymorphism identified 24 genes with polymorphic tetranucleotide repeats. For selecting genes with trinucleotide repeats, we used a database published by Kozlowski et al, ²² where 878 genes with more than 6 units of trinucleotide repeats are listed. Among them, we selected 64 polymorphic genes with more than 8 units of trinucleotide repeats in their 5′-UTR, an exon, or 3′-UTR. To select a gene with dinucleotide repeats, we used the Sate/log Database. ²³ We selected 45 genes containing polymorphic CA/GT with 8 or more units (8-49 units) in their 5′UTR, an intron or 3′UTR (Tables 5.1, 5.2, and 5.3).

TABLE 5.1
GENE WITH DINUCLEOTIDE REPEATS (45 GENES)

| Gene | Cancer | Repeats | No. Unit | Position |
|---|---|---|---|---|
| MNT | Y | CA | 23 | 3′ |
| HEC1 | Y | CA | 27 | intron |
| SEMA6D | Y | CA | 22 | 3′ |
| FGF3 | Y | CA | 29 | 3′ |
| MLH3 | Y | CA | 13 | intron |
| IGF1 | Y | CA | 22 | 3′ |
| PAX5 | Y | CA | 21 | 3′ |
| ZEB1 | Y | CA | 19 | 3′ |
| PTP4A2 | Y | CA | 25 | 3′ |
| SNX20 | Y | CA | 21 | 3′ |
| CNOT3 | Y | CA | 18 | 5′ |
| PHF17 | Y | CA | 23 | 3′ |
| STYK1 | Y | CA | 11 | 3′ |
| MAPKAPK2 | Y | CA | 15 | 3′ |
| NDRG4 | Y | CA | 14 | 3′ |
| AKAP11 | Y | CA | 21 | 3′ |
| MAP2 | Y | CA | 16 | 3′ |
| BMP4 | Y | CA | 16 | 3′ |
| RDX | Y | CA | 15 | 3′ |
| SATB1 | Y | CA | 18 | 3′ |
| FASLG | Y | CA | 15 | 3′ |
| MACC1 | Y | CA | 49 | 3′ |
| NLK | Y | CA | 16 | 3′ |
| PTGES | Y | CA | 24 | 3′ |
| HIF1β | Y | CA | 15 | 3′ |
| MAPK10 | Y | CA | 16 | 3′ |
| EFNB1 | Y | CA | 14 | 3′ |
| UBLCP1 | N | CA | 24 | 3′ |
| ATF1 | Y | CA | 8 | 3′ |
| SNAIL2 | Y | CA | 15 | 3′ |
| SMAD7 | Y | CA | 11 | 3′ |
| ENOS | Y | CA | 34 | intron |
| PTPRT | Y | CA | 25 | intron |
| DUSP10 | Y | CA | 20 | intron |
| MSH3 | Y | CA | 20 | intron |
| TMPRSS2 | Y | CA | 21 | intron |
| PTEN | Y | CA | 19 | intron |
| EGFR | Y | CA | 16 | intron |
| MSH6 | Y | CA | 17 | intron |
| DEC1 | Y | CA | 24 | intron |
| LMO1 | Y | CA | 15 | 5′ |
| BMP3 | Y | CA | 17 | 5′ |
| CLEC2B | Y | CA | 10 | 5′ |
| XPO5 | Y | CA | 24 | intron |
| MAF | Y | CA | 23 | 3′ |

TABLE 5.2
GENE WITH TRINUCLEOTIDE REPEATS (64 GENES)

| Gene | Cancer | Repeats | No. Unit | Position |
|---|---|---|---|---|
| WIPF2 | Y | CGG | 9 | 5′ |
| KDM6B | Y | CCA | 12 | exon |
| SMARCA2 | Y | CAG | 20 | exon |
| BCL6B | Y | CAG | 9 | exon |
| NADK | N | GGA | 8 | exon |
| UBE2B | Y | CGG | 10 | 5′ |
| PRKCSH | N | GAG | 19 | exon |
| KCNN3 | Y | CAG | 13, 14 | exon |
| GABRA4 | N | AAT | 14 | 3′ |
| MTMR9 | N | GTT | 8 | 3′ |
| DMPK | Y | CAG | 20 | 3′ |
| GRIK2 | Y | AAT | 14 | 3′ |
| PDCD1 | Y | CAG | 10 | 3′ |
| SPRY4 | Y | AAC | 10 | 3′ |
| PRDM10 | N | AAG | 10 | 3′ |
| HERC5 | N | AAC | 9 | 3′ |
| ARCN1 | N | ATT | 9 | 3′ |
| ZNF516 | N | AAC | 9 | 3′ |
| ZNF790 | N | CAG | 9 | 3′ |
| SIRPA | Y | ACC | 9 | 3′ |
| RREB1 | Y | ATT | 8 | 3′ |
| VKORC1L1 | N | ATT | 8 | 3′ |
| NRP2 | Y | TAT | 10 | 3′ |
| VANGL2 | Y | ATT | 10 | 3′ |
| CRKL | Y | AAC | 9 | 3′ |
| HOXB6 | Y | GAT | 9 | 3′ |
| PAPSS2 | Y | CAG | 8 | 5′ |
| BLMH | Y | CCG | 9 | 5′ |
| MTHFD1L | N | CCG | 8 | 5′ |
| MAB21L1 | N | CAG | 19 | 5′ |
| GLS | Y | CAG | 15 | 5′ |
| STRC | N | CAG | 11 | 5′ |
| GRK5 | Y | CGG | 9 | 5′ |
| GALNT5 | N | CAG | 8 | 5′ |
| BPGM | N | CAG | 8 | 5′ |
| TRHDE | N | AGG | 8 | 5′ |
| MAF2 | Y | CGG | 8 | 5′ |
| PCTK3 | Y | AGG | 8 | 5′ |
| STC1 | Y | CAG | 6 | 3′ |
| YEATS2 | N | GGA | 9 | exon |
| TNRC6B | Y | CAG | 8 | exon |
| DACH1 | Y | CAG | 24 | exon |
| NKD2 | Y | CAC | 9 | exon |
| ASPN | N | TGA | 14 | exon |
| ATBF1 | Y | GGA | 24 | exon |
| C19ORF2 | Y | TGA | 9 | exon |
| CHAC | Y | TGA | 11 | exon |
| DIAPH1 | Y | GGA | 11 | exon |
| EPHB6 | Y | AGG | 8 | exon |
| CBX4 | N | GTG | 11 | exon |
| C14ORF4 | N | CAG | 21 | exon |
| HRC | N | GAT | 13 | exon |
| SNAPC4 | N | GCA | 9 | exon |
| HTT | N | GCC | 10 | exon |
| NCOA3 | Y | GCA | 20 | exon |
| BMP2K | N | CAG | 27 | exon |
| MN1 | Y | CAG | 27 | exon |
| ZNF384 | Y | CAG | 16 | exon |
| BAIAP1 | N | CAG | 20 | exon |
| SCA1 | Y | CAG | 29 | exon |
| NCOR2 | Y | CAG | 12 | exon |
| GATA6 | Y | CCA | 10 | exon |
| PLCZ1 | Y | GGA | 15 | exon |
| ZNF161 | N | CAG, CAA | 12, 6 | exon |

TABLE 5.3
GENE WITH TETRANUCLEOTIDE REPEATS (33 GENES)

| | | | No. | |
|---|---|---|---|---|
| Gene | Cancer | Repeats | Unit | Position |
| SLC5A12 | Y | AAAG | 13 | 3′ |
| RBM47 | N | AAAG | 16 | 3′ |
| FZD4 | Y | CAAA | 8 | 3′ |
| ANKRD5 | N | ATGA, TAGA | 5, 10 | 3′ |
| BCL2(D18S51) | Y | AAAG | 18 | intron |
| HDAC4 | Y | TAGA | 9 | 3′ |
| KCNK2 | Y | TAGA | 13 | 3′ |
| D5S818 | N.A. | AGAT | 11 | intergenic |
| D13S317 | N.A. | TATC | 11 | intergeneic |
| KMO | N | TAGA | 19 | 3′ |
| D21S11 | N.A. | TAGA | 11 | intergenic |
| DAP3 | Y | AAAT | 10 | 3′ |
| TBX19 | Y | AAAG | 6 | 3′ |
| SHROOM4 | N | AAAG | 15 | 3′ |
| ORC6L | Y | TAGA | 13 | 3′ |
| TPOX | N.A. | TGAA | 8 | intron |
| NHLH1 | Y | TTTA | 13 | 3′ |
| C20ORF56 | N | AAAG | 14 | 3′ |
| PMP2 | N | TAGA | 14 | 3′ |
| SNX1 | Y | GATA | 16 | 3′ |
| D2S1338 | N.A. | AAGG | 13 | intergenic |
| D9S303 | N.A. | GATA | 12 | intron |
| SNX27 | N | AAAG | 20 | 3′ |
| D8S1179 | N.A. | TAGA | 11 | intron |
| PLEKHG4B | N | TAGA | 10 | 3′ |
| KANK2 | N | GGAT | 13 | 3′ |
| PLCXD3 | N | GGAA | 12 | 3′ |
| ZFR2 | N | CAAA | 9 | 3′ |
| RTKN2 | N | AAGG | 16 | 3′ |
| C19orf2(D19S433) | N.A. | AGGA | 13 | intron |
| CDH1 | Y | AAAG | 20 | intron |
| MOG | N | AAAT | 11 | 3′ |
| FGA | N | AAAG | 14 | intron |

The association of a selected gene with “cancer” was examined by accessing the NCBI PubMed literature database (www.ncbi.nlm.nih.gov/pubmed). Seventy percent of the selected genes have been reported to be associated with cancer in literature. In addition to the above genes, we added the 9 EMAST markers that were frequently mutated in cancer tissues to the list.¹⁵ Template DNA from 24 cases of LM tissues that exhibited MSI-M and matched normal tissues were amplified for each of these loci and analyzed for MSI and LOH.

SMARCA2R LOH. Four polymorphic markers SMARCA2-2, SMARCA2-4, SMARCA2-230K and SMARCA2-240K were used to detect LOH from the approximately 300 Kb region spanning the SMARCA2 locus. The primer sequences for these loci are as follows: SMARCA2-2-F (5′-TGTAAAACGACGGCCAGTAGGGGAAAAGGACGTTGC-3′) (SEQ ID NO: 1), SMARCA2-2-R (5′-TGTTGTTGCTGCGTCTGTG-3′) (SEQ ID NO: 2), SMARCA2-4-F (5′-TGTAAAACGACGGCCAGTAGCCTGAACACTGCATAGTGAG-3′) (SEQ ID NO: 3) SMARCA2-4-R (5′-TCATCTTTTGGAAATGGAATAAGG-3′) (SEQ ID NO: 4), SMARCA2-230K-F (5′-GAAACATAACCAAGAAGATGGATG-3′) (SEQ ID NO: 5), SMARCA2-230K-R (5′-TGTAAAACGACGGCCAGTCCAGCTTCTGCAATGGTGTA-3′) (SEQ ID NO: 6), SMARCA2-240K-F 5′-TTTTTAAACAGCCCAACTTTCA-3′) (SEQ ID NO: 7) and SMARCA2-240K-R (5′-CACACCCACTTTTCAGAGGA-3′) (SEQ ID NO: 8). LOH was defined as positive when one of the four markers showed LOH, and as not informative when homozygousity was detected in all three markers. The remainder of the cases was defined as non-LOH.

Statistical Analysis. The Chi-square test and multiple logistic regression analyses were used for assessing the association of MSI-M with clinicopathological factors. To estimate recurrence-free survival for a particular group of CRC, the Kaplan-Meier method was used. To evaluate significant differences between groups, the log rank test was used. The Cox proportional hazards regression analysis was used to evaluate the association between MSI-M and other clinicopathological factors for predicting recurrent distant metastasis. If a P value was less than 0.05, the difference was considered to be statistically significant.

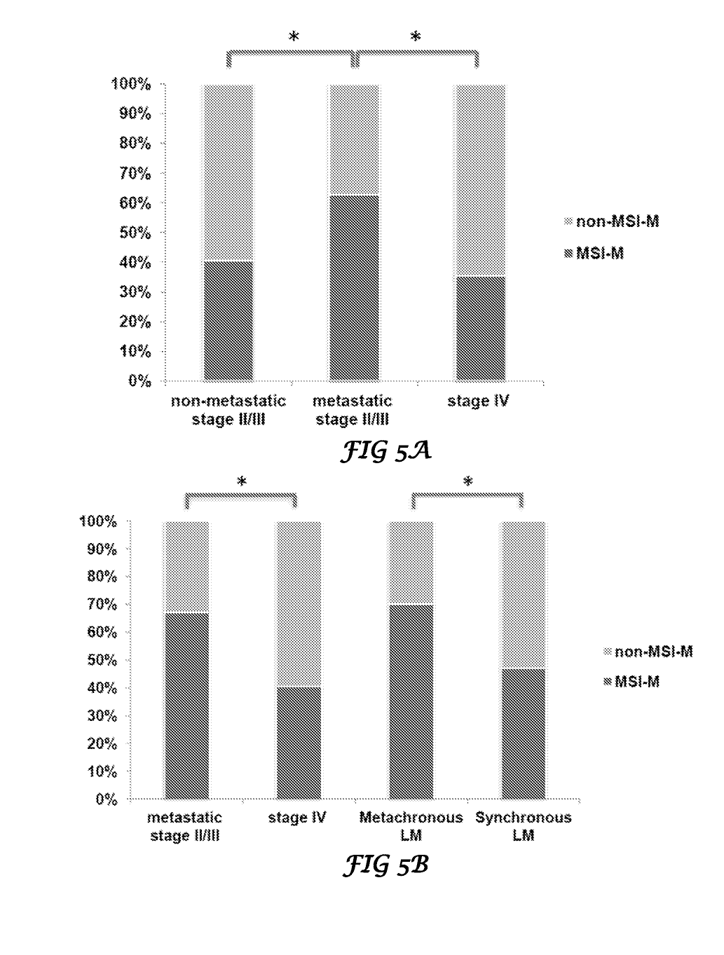

MSI-M in primary CRC and the liver metastasis from CRC. The example above examined. 167 cases of primary CRC for microsatellite mutations at 7 referenced NCI microsatellite loci and 7 EMAST loci.²⁰ Among 167 tumors, 42 cases were stage primary CRCs that did not give rise to recurrent distant metastasis within 60 months after the initial diagnosis, 56 cases were stage II/III primary CRCs that gave rise to distant metastasis within 60 months after diagnosis, and 17 cases were stage IV primary CRC that were associated with synchronous metastasis. As shown in FIG. 5A, MSI-M was more frequently observed in metastatic (62.5%, 35 of 56 cases) than in non-metastatic stage primary CRC (35.3%, 17 of 42 cases) in stage IV CRC (40.5%, 6 of 17 cases); differences were significant in each case (P=0.031 and P=0.048 respectively). In contrast, there was no difference in frequency of MSI-M between non-metastatic stage II/III primary CRC (35.3%) and stage IV CRC (40.5%, 6 of 17 cases) (P=0.712).

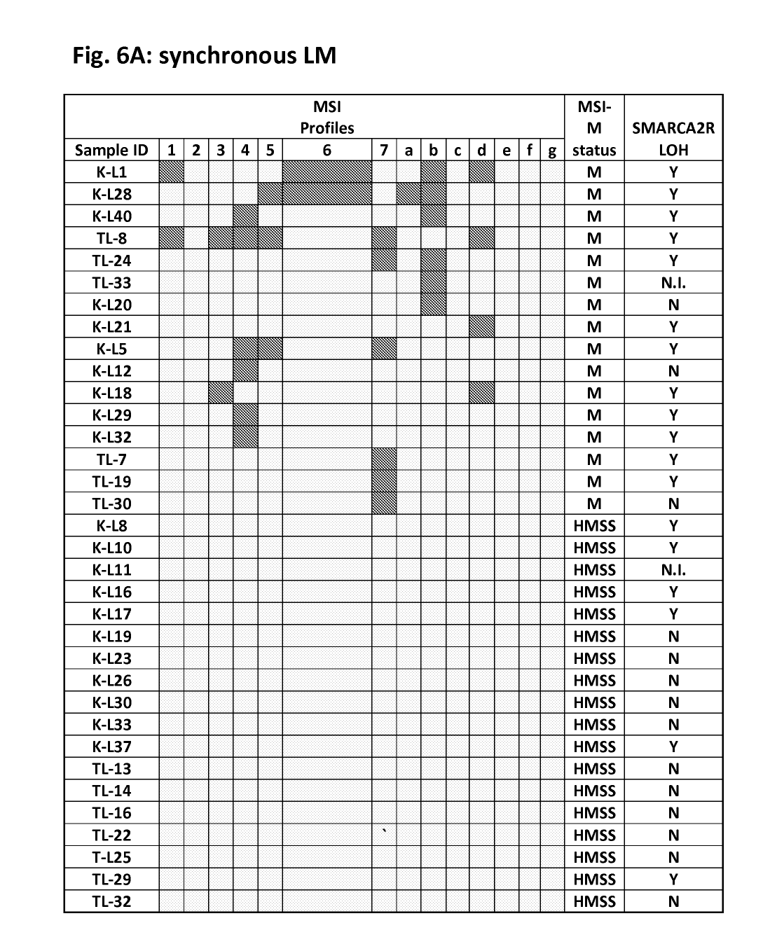

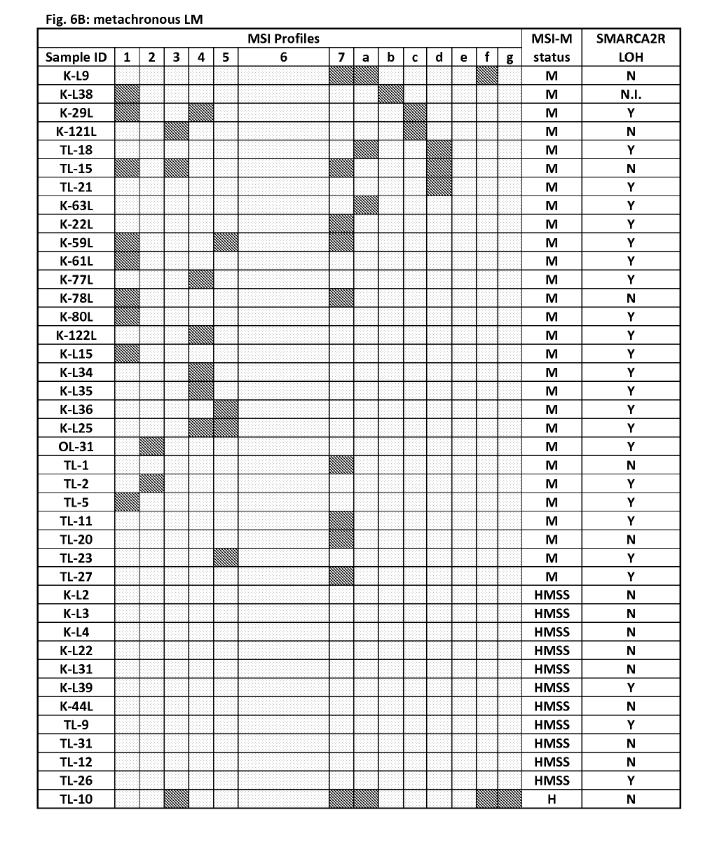

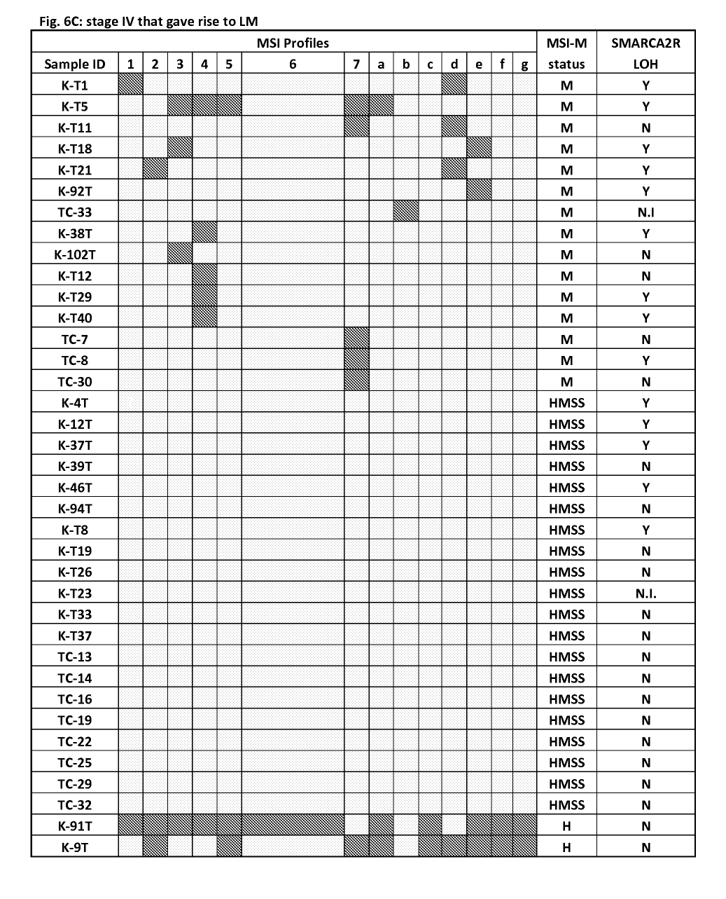

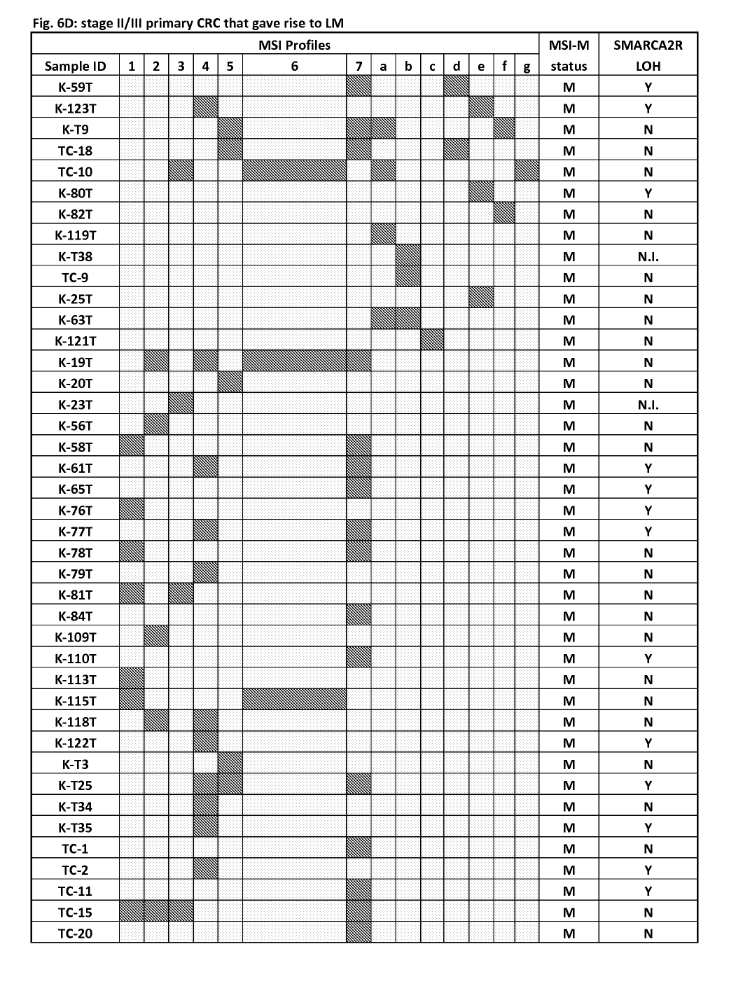

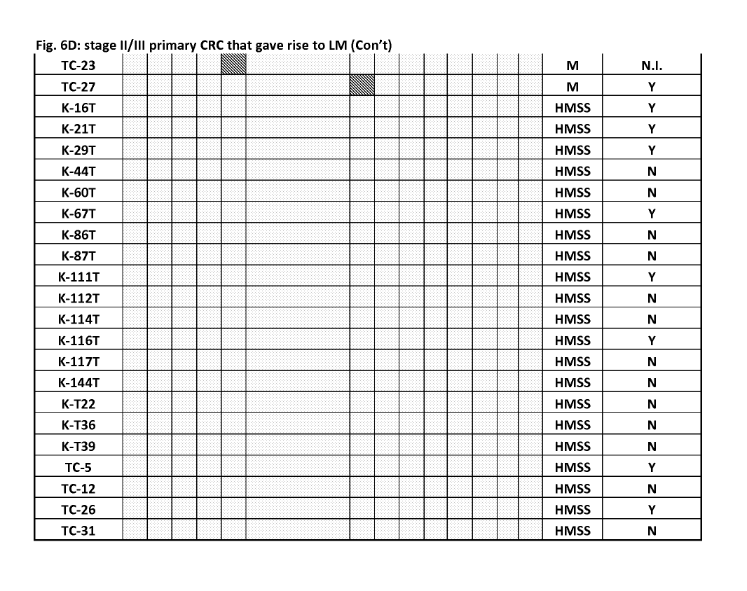

Because MSI-M is associated with higher risk for recurrent metastasis than non-MSI-M tumors in stage II/III CRC,²⁰ it would be expected to see a higher frequency of MSI-M in metachronous metastasis tissues from primary CRC if MSI status does not change after dissemination. To examine how prevalent MSI-M is in LM from primary CRC, the MSI status of 74 LM tissues including 34 synchronous and 40 metachronous LM (FIG. 6A to 6D) was determined. White 47.1% of synchronous LM (16/34 cases) showed MSI-M (FIG. 1B, Table 6), 70.0% of metachronous LM (28 of 40 cases) showed MSI-M. This difference was statistically significant (FIG. 5B, Table 6, P=0.045).

TABLE 6
MSI-M is enriched in metachronous LM compared to synchronous LM.

| | No. of | | No. of | | |
|---|---|---|---|---|---|
| Factors | synchronous LM (%) | | metachronous LM (%) | | P values |
| Age | | | | | |
| ≦62 | 14 | (41.2) | 25 | (62.5) | |
| >62 | 20 | (58.8) | 15 | (37.5) | 0.067 |
| Sex | | | | | |
| F | 11 | (32.4) | 13 | (32.5) | |
| M | 23 | (67.6) | 26 | (67.5) | 0.929 |
| Grade^{c} | | | | | |
| G1 | 4 | (11.8) | 17 | (42.5) | |
| G2 + G3 | 30 | (88.2) | 23 | (57.5) | 0.003 |
| Location^{d} | | | | | |
| Proximal | 6 | (17.7) | 3 | (7.5) | |
| Distal | 28 | (82.3) | 37 | (92.5) | 0.183 |
| MSI | | | | | |
| non-MSI-M | 18 | (52.9) | 12 | (30.0) | |
| MSI-M | 16 | (47.1) | 28 | (70.0) | 0.045 |
| Population | | | | | |
| Japanese | 12 | (35.3) | 17 | (42.5) | |
| Korean | 22 | (64.7) | 23 | (57.5) | 0.527 |
| Total | 34 | | 40 | | |

^{a}P values were determined by chi square test.
^{b}A degree of differentiation exhibited by primary CRCs from which the LMs originated. G1: well differentiated, G2: moderatly diffrentiated, G3: poorly differentiated.
^{c}A location of primary CRCs from which the LMs originated. Proximal includes cecum ascending and traverse colon. Distal includes sigmoid colon and rectal.

The MSI status of 49 primary CRC that gave rise to LM (FIG. 6A to 6D) was examined. The data for 52 cases of primary CRC that gave rise to LM (hereinabove) were also added to the analysis. In total the MSI status of 101 such cases was determined. Among them, 37 cases were stage IV (FIG. 6C) and 64 cases were stage II/III (FIG. 5B and FIG. 6D). 40.5% of stage IV CRC (15 of 37 cases) exhibited MSI-M while 67.2% of stage II/III primary CRC that gave rise to LM (43 of 64 cases) were positive for MSI-M; this difference was significant (P=0.01) (FIG. 5B, Table 6).

TABLE 7
MSI-M is enriched in primary II and III that gave rise to LM.

| | No. of primary | | No. | | |
|---|---|---|---|---|---|
| Factors | II and III (%) | | of primary IV (%) | | P values |
| Age | | | | | |
| ≦62 | 38 | (59.4) | 20 | (54.1) | |
| >62 | 26 | (40.6) | 17 | (45.9) | 0.602 |
| Sex | | | | | |
| F | 22 | (34.4) | 14 | (37.8) | |
| M | 42 | (65.6) | 23 | (62.2) | 0.726 |
| Grade^{c} | | | | | |
| G1 | 22 | (34.4) | 6 | (16.2) | |
| G2 + G3 | 42 | (65.6) | 31 | (83.2) | 0.05 |
| Location^{d} | | | | | |
| Proximal | 7 | (10.9) | 8 | (21.6) | |
| Distal | 57 | (89.1) | 31 | (76.4) | 0.181 |
| MSI | | | | | |
| non-MSI-M | 21 | (32.8) | 22 | (59.5) | |
| MSI-M | 43 | (67.2) | 15 | (40.5) | 0.01 |
| Population | | | | | |
| Japanese | 13 | (20.3) | 12 | (32.4) | |
| Korean | 51 | (79.7) | 25 | (67.6) | 0.248 |
| Total | 64 | | 37 | | |

^{a}P values were determined by chi square test.
^{b}A degree of differentiation exhibited by primary CRCs from which the LMs originated. G1: well differentiated, G2: moderatly diffrentiated, G3: poorly differentiated.
^{c}A location of primary CRCs from which the LMs originated. Proximal includes cecum ascending and traverse colon. Distal includes sigmoid colon and rectal.

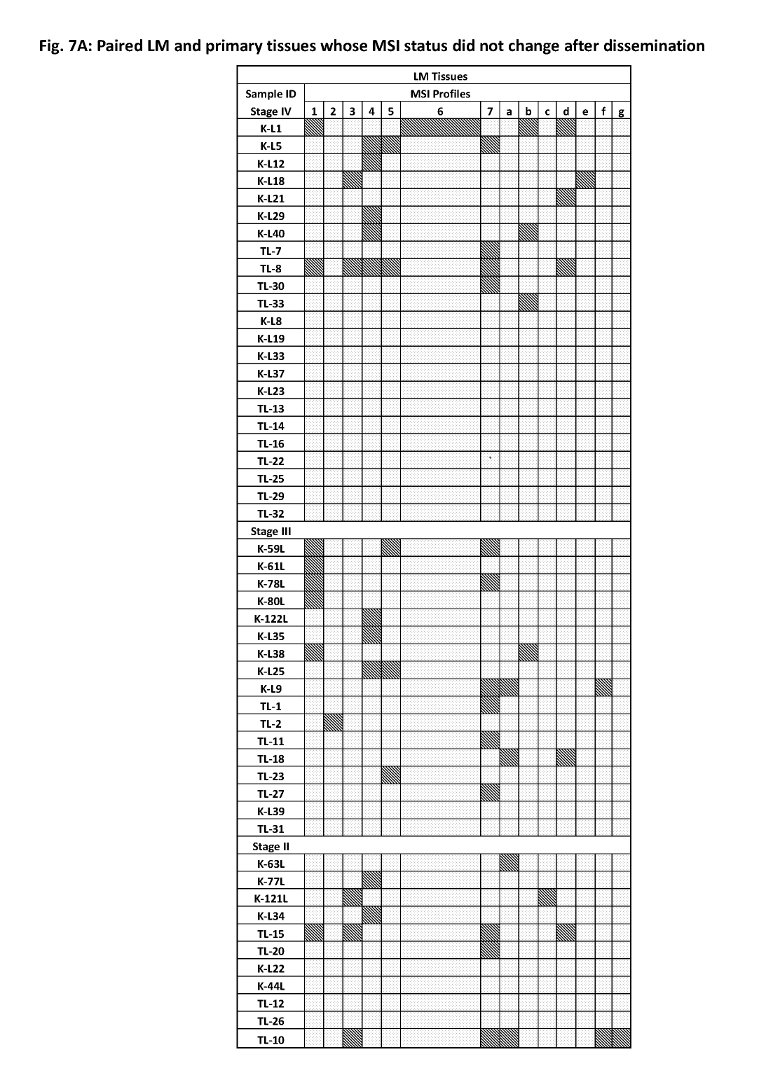

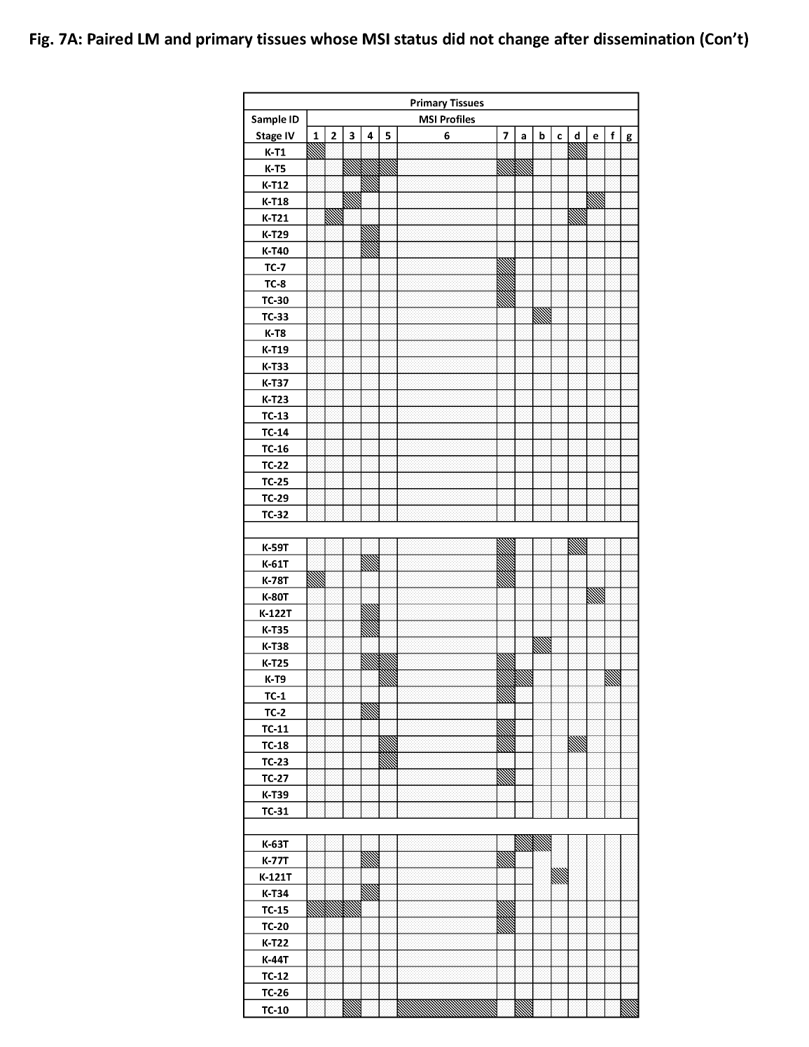

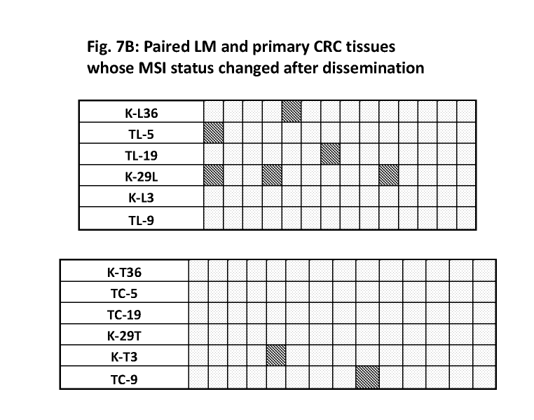

As shown in FIG. 5B, there was no significant change in the frequencies of MSI-M between primary CRC that gave rise to LM and LM tissues. This was confirmed when the MSI status of the 63 matched LMs and primary CRCs from which these LMs originated were compared. MSI status changed in only 6 matched cases (9.5%), 4 cases where MSI status changed from MSS to MSI-M and 2 cases where MSI status changed from MSI-M to MSS after dissemination. Thus, the MSI status of primary CRC reflects that of metastasized tissues in most cases (˜90%) (FIGS. 7A and 7B).

FIGS. 7A and 7B show MSI profiles between paired LM and corresponding primary CRC. FIGS. 7A and 7B provide a detailed data for MSI profiles between LM and corresponding primary CRC from which the LM was derived. FIG. 7A: Fifty-one pairs whose MSI profiles were similar to each other. FIG. 7B: Six pairs whose MSI profiles changed after dissemination. The columns depict the following: mutation data for 7 EMAST markers (1 through 7), 5 markers with CA repeats (a through e), 2 markers with mono-A repeats (f and g). A green box indicates the presence of a frame-shift mutation. Each number corresponds to EMAST and letter corresponds to NO markers as follows: 1: MYCL1, 2:D19S394, 3:D20S85, 4: D20S82, 5: D9S242, 6: L17835, 7: D8S321, a: D2S123, b: D175250, c: D5S346, d: D18S64, e: D18569, f: BAT25, g: BAT26.

Taken together, these results indicate that MSI-M was significantly associated with stage primary CRC that gave rise distant metastasis including metastasis to the liver. A significant association with MSI-M was also detected in metachronous LM. These results are compatible with the finding hereinabove that MSI-M is an independent predictor of stage II/III primary CRC for recurrent distant metastasis. However, it was not known how MSI-M links to recurrent distant metastasis in CRC.

One possibility is that MSI-M CRC could be more tolerant to 5-FU treatment than is H-MSS or MSI-H CRC. This assumption comes from the above observation that MSI-M is enriched in metachronous LM compared to synchronous LM (FIG. 5B) and the fact that most of the precursors of metachronous LM but not those of synchronous LM were exposed to 5-FU based adjuvant chemotherapy. In fact, among our 64 cases of metachronous LM, 82.4% (14 of 17 cases) of stage II primary and 85.1% (40 of 47 cases) of stage III primary CRC corresponding to these LM cases had received 5-FU based adjuvant chemotherapy. Thus, a higher frequency of MSI-M in metachronous LM might reflect its precursor's resistance to 5-FU exposure. However, this may not be the case for the following two reasons. First multivariable logistic regression analysis for 48 cases of metachronous LM analyzed in this study failed to detect any significant association between prior treatment of primary CRC with 5-FU and MSI-M exhibited by the metachronous LM (P=0.5205). Second, the studies hereinabove showed that MSI-M exhibited by stage II/III primary CRC is an independent predictor for recurrence regardless of adjuvant chemotherapy. ²⁰

Screening of a gene(s) with microsatellites that is associated with MSI-M. To determine how MSI-M links to distant metastasis, a genetic alteration associated with MSI-M and with the ability to metastasize to the liver was identified. First, 142 candidate genes containing di-, tri- or tetranucleotide repeats in intragenic sequences (Tables 5.1 to 5.3) were selected. These genes were screened for high frequencies of MSI or LOH in 24 cases of LM that had been found to be positive for MSI-M in the studies described above.

Among 142 gene loci examined, 29 loci (20.4%) exhibited MSI in 24 cases of MSI-M-positive LM (Tables 8.1 and 8.2).

TABLE 8.1
MSI Genes

| | | | | Repeats | Mutation |
|---|---|---|---|---|---|
| Genes | Cancer | Repeats | No. Unit | Position | Freq. (%) |
| RBM47 | N | AAAG | 16 | 3′ | 6/24 (25) |
| WIPF2 | Y | CGG | 9 | 5′ | 4/22 (18) |
| D9S303 | N.A. | GATA | 12 | intron | 4/24 (17) |
| ZNF161 | N | CAG, CAA | 12, 6 | exon | 4/24 (17) |
| D8S1179 | N.A. | TAGA | 11 | intron | 3/21 (14) |
| D21S11 | N.A. | TAGA | 11 | intergenic | 3/24 (13) |
| KANK2 | N | GGAT | 13 | 3′ | 3/24 (13) |
| DAP3 | Y | AAAT | 10 | 3′ | 2/23 (9) |
| MOG | N | AAAT | 11 | 3′ | 2/22 (9) |
| KCNN3 | Y | CAG | 13, 14 | exon | 2/24 (8) |
| DACH1 | Y | CAG | 24 | exon | 1/12 (8) |
| SCA1 | Y | CAG | 29 | exon | 2/24 (8) |
| KMO | N | TAGA | 19 | 3′ | 2/24 (8) |
| D2S1338 | N.A. | AAGG | 13 | intergenic | 2/24 (8) |
| CDH1 | Y | AAAG | 20 | intron | 2/24 (8) |
| XPO5 | Y | CA | 24 | intron | 2/24 (8) |
| RTKN2 | N | AAGG | 16 | 3′ | 1/20 (5) |
| LMO1 | Y | CA | 15 | 5′ | 1/24 (4) |
| PRKCSH | N | GAG | 19 | exon | 1/23 (4) |
| PAPSS2 | Y | CAG | 8 | 5′ | 1/23 (4) |
| TNRC6B | Y | CAG | 8 | exon | 1/24 (4) |
| NKD2 | Y | CAC | 9 | exon | 1/23 (4) |
| SLC5Al2 | Y | AAAG | 13 | 3′ | 1/24 (4) |
| FZD4 | Y | CAAA | 8 | 3′ | 1/23 (4) |
| BCL2(D18S51) | Y | AAAG | 18 | intron | 1/24 (4) |
| TPOX | N.A. | TGAA | 8 | intron | 1/24 (4) |
| C20ORF56 | N | AAAG | 14 | 3′ | 1/24 (4) |
| SNX1 | Y | GATA | 16 | 3′ | 1/23 (4) |
| C19orf2(D19S433) | N.A. | AGGA | 13 | intron | 1/24 (4) |

TABLE 8.2
LOH Genes

| | | | No. | Repeats | Mutation |
|---|---|---|---|---|---|
| Genes | Cancer | Repeats | Unit | Position | Freq. (%) |
| KDM6B | Y | CCA | 12 | exon | 6/8 (75) |
| MNT | Y | CA | 23 | 3′ | 12/17 (71) |
| SMARCA2 | Y | CAG | 20 | exon | 7/11 (64) |
| HEC1 | Y | CA | 27 | intron | 6/10 (60) |
| ANKRD5 | N | ATGA, TAGA | 5, 10 | 3′ | 7/12 (58) |
| BCL2(D18S51) | Y | AAAG | 18 | intron | 11/19 (58) |
| SEMA6D | Y | CA | 22 | 3′ | 8/14 (57) |
| D5S818 | N.A. | AGAT | 11 | intergenic | 9/16 (56) |
| STYK1 | Y | CA | 11 | 3′ | 6/12 (50) |
| BCL6B | Y | CAG | 9 | exon | 3/6 (50) |
| ZNF516 | N | AAC | 9 | 3′ | 8/17 (47) |
| KCNK2 | Y | TAGA | 13 | 3′ | 6/13 (46) |
| RBM47 | N | AAAG | 16 | 3′ | 6/14 (43) |
| MOG | N | AAAT | 11 | 3′ | 3/7 (43) |
| CLEC2B | Y | CA | 10 | 5′ | 6/14 (43) |
| FGF3 | Y | CA | 29 | 3′ | 8/19 (42) |
| PRDM10 | N | AAG | 10 | 3′ | 6/15 (40) |
| ORC6L | Y | TAGA | 13 | 3′ | 3/8 (38) |
| PLCZ1 | Y | GGA | 15 | exon | 3/8 (38) |
| PLCXD3 | N | GGAA | 12 | 3′ | 7/19 (37) |
| MLH3 | Y | CA | 13 | intron | 5/14 (36) |
| KMO | N | TAGA | 19 | 3′ | 6/17 (35) |
| MAF | Y | CGG | 8 | 5′ | 6/17 (35) |
| MAF | Y | CA | 23 | 3′ | 6/17 (35) |
| NADK | N | GGA | 8 | exon | 3/9 (33) |
| UBE2B | Y | CGG | 10 | 5′ | 2/6 (33) |
| PRKCSH | N | GAG | 19 | exon | 3/9 (33) |
| PDCD1 | Y | CAG | 10 | 3′ | 1/3 (33) |
| NCOA3 | Y | GCA | 20 | exon | 3/9 (33) |
| PAX5 | Y | CA | 21 | 3′ | 4/12 (33) |
| SNX20 | Y | CA | 21 | 3′ | 4/12 (33) |
| SATB1 | Y | CA | 18 | 3′ | 1/3 (33) |
| PTEN | Y | CA | 19 | intron | 4/12 (33) |
| DEC1 | Y | CA | 24 | intron | 5/15 (33) |
| HIF1β | Y | CA | 15 | 3′ | 4/12 (33) |
| XPO5 | Y | CA | 24 | intron | 1/3 (33) |
| SNX1 | Y | GATA | 16 | 3′ | 4/13 (31) |
| RDX | Y | CA | 15 | 3′ | 3/10 (30) |
| HDAC4 | Y | TAGA | 9 | 3′ | 4/13 (31) |
| RTKN2 | N | AAGG | 16 | 3′ | 4/14 (29) |
| PCTK3 | Y | AGG | 8 | 5′ | 4/14 (29) |
| HRC | N | GAT | 13 | exon | 5/17 (29) |
| NCOR2 | Y | CAG | 12 | exon | 5/17 (29) |
| FGA | N | AAAG | 14 | intron | 4/15 (27) |
| C19orf2(D19S433) | N.A. | AGGA | 13 | intron | 5/19 (26) |
| PTGES | Y | CA | 24 | 3′ | 5/19 (26) |
| LMO1 | Y | CA | 15 | 5′ | 5/19 (26) |
| NDRG4 | Y | CA | 14 | 3′ | 2/8 (25) |
| DIAPH1 | Y | GGA | 11 | exon | 1/4 (25) |
| C14ORF4 | N | CAG | 21 | exon | 1/4 (25) |
| BLMH | Y | CCG | 9 | 5′ | 2/8 (25) |
| MACC1 | Y | CA | 49 | 3′ | 1/4 (25) |
| MTMR9 | N | GTT | 8 | 3′ | 2/8 (25) |
| FZD4 | Y | CAAA | 8 | 3′ | 3/12 (25) |
| D13S317 | N.A. | TATC | 11 | intergeneic | 5/21 (24) |
| CNOT3 | Y | CA | 18 | 5′ | 4/17 (24) |
| KCNN3 | Y | CAG | 13, 14 | exon | 3/13 (23) |
| D21S11 | N.A. | TAGA | 11 | intergenic | 4/18 (22) |
| ATBF1 | Y | GGA | 24 | exon | 4/18 (22) |
| SNX27 | N | AAAG | 20 | 3′ | 1/5 (20) |
| GABRA4 | N | AAT | 14 | 3′ | 2/10 (20) |
| NRP2 | Y | TAT | 10 | 3′ | 3/15 (20) |
| BAIAP1 | N | CAG | 20 | exon | 2/10 (20) |
| ZEB1 | Y | CA | 19 | 3′ | 4/20 (20) |
| PHF17 | Y | CA | 23 | 3′ | 4/21 (19) |
| D9S303 | N.A. | GATA | 12 | intron | 2/11 (18) |
| CDH1 | Y | AAAG | 20 | intron | 3/17 (18) |
| YEATS2 | N | GGA | 9 | exon | 2/12 (17) |
| VANGL2 | Y | ATT | 10 | 3′ | 1/6 (17) |
| DMPK | Y | CAG | 20 | 3′ | 2/12 (17) |
| SLC5A12 | Y | AAAG | 13 | 3′ | 2/13 (15) |
| PLEKHG4B | N | TAGA | 10 | 3′ | 2/13 (15) |
| VKORC1L1 | N | ATT | 8 | 3′ | 2/13 (15) |
| TPOX | N.A. | TGAA | 8 | intron | 2/14 (14) |
| MTHFD1L | N | CCG | 8 | 5′ | 1/7 (14) |
| MAPKAPK2 | Y | CA | 15 | 3′ | 1/7 (14) |
| GLS | Y | CAG | 15 | 5′ | 2/17 (12) |
| TBX19 | Y | AAAG | 6 | 3′ | 2/19 (11) |
| SCA1 | Y | CAG | 29 | exon | 2/19 (11) |
| PTP4A2 | Y | CA | 25 | 3′ | 2/20 (10) |
| MAP2 | Y | CA | 16 | 3′ | 1/10 (10) |
| NLK | Y | CA | 16 | 3′ | 1/11 (9) |
| C20ORF56 | N | AAAG | 14 | 3′ | 1/13 (8) |
| IGF1 | Y | CA | 22 | 3′ | 1/13 (8) |
| ASPN | N | TGA | 14 | exon | 1/15 (7) |
| PTPRT | Y | CA | 25 | intron | 1/14 (7) |
| D8S1179 | N.A. | TAGA | 11 | intron | 1/17 (6) |

As expected,¹⁶ more loci with larger repeats showed NISI than loci with smaller repeats; 53% of loci with tetranucleotide repeats, 15% of loci with trinucleotide repeats and 4% of loci with di-nucleotide repeats showed MSI. As shown in Table 1, RBA/147 (25%), WIPF (18%), D9S303 (17%), ZNF161 (17%), D8S1179 (14%), D21S11 (13%) and KANK2 (13%) exhibited higher levels of MSI in their microsatellite regions in MSI-M-positive LMs. However, the mutation frequency of these loci was no greater than the average mutation frequency of 7 EMAST markers (˜20%) among 24 LM cases. Although none of these loci has been associated with cancer in literature by PubMed search, it remains to be determined whether MSI in these loci has any biological function, or has relationship to MSI-M and metastasis.

TABLE 9
Gene Loci frequently shows MSI or LOH in 24 cases of MSI-M positive LM.

| | | | | Repeats | Mutation | |
|---|---|---|---|---|---|---|
| Genes | Cancer^{a} | Repeats | No. Unit | Position | Freq. (%)^{b} | Gene Location |
| (MSI) | | | | | | |
| RBM47 | N | AAAG | 16 | 3′ | 6/24 (25) | 4p14 |
| WIPF2 | N | CGG | 9 | 5′ | 4/22 (18) | 17q21 |
| D9S303 | N.A. | GATA | 12 | intron | 4/24 (17) | 8q21.32 |
| ZNF161 | N | CAG, CAA | 12, 6 | exon | 4/24 (17) | 17q22 |
| D8S1179 | N.A. | TAGA | 11 | intron | 3/21 (14) | 8q24.13 |
| D21S11 | N.A. | TAGA | 11 | intergenic | 3/24 (13) | 21q21.1 |
| KANK2 | N | GGAT | 13 | 3′ | 3/24 (13) | 19p13.2 |
| (LOH) | | | | | | |
| KDM6B | Y | CCA | 12 | exon | 6/8 (75) | 17p13.1 |
| MNT | Y | CA | 23 | 3′ | 12/17 (71) | 17p13.3 |
| SMARCA2 | Y | CAG | 20 | exon | 7/11 (64) | 9p24.3 |
| HEC1 | Y | CA | 27 | intron | 6/10 (60) | 18p11.32 |
| ANKRD5 | N | ATGA, TAGA | 5, 10 | 3′ | 7/12 (58) | 20p12.2 |
| BCL2 (D18S51) | Y | AAAG | 18 | intron | 11/19 (58) | 18p21.33 |
| SEMA6D | Y | CA | 22 | 3′ | 8/14 (57) | 15p21.1 |
| D5S818 | N.A. | AGAT | 11 | intergenic | 9/16 (56) | 5q23.2 |
| STYK1 | Y | CA | 11 | 3′ | 6/12 (50) | 12p13.2 |
| BCL6B | Y | CAG | 9 | exon | 3/6 (50) | 17p13.1 |

^{a}Each gene locus was examined for the association with cancer by accessing NCBI Pubmed data base. Y: the locus has been associated with cancer; N: no association has been reported. N.A. not applicable.
^{b}A mutation frequency was determined by ratio between the number of mutated cases divided by the number of informative cases.

Compared to MSI, LOH was found in more loci with higher frequencies. Eighty-seven out of 142 loci (61%) exhibited LOFT with more than 6% of informative cases (Table 8). These results suggest that a large number of genetic alterations in MSI-M-positive LM may be generated through a chromosome instability pathway associated with LOH even though these tumors exhibit moderate levels of MSI.

The present inventors found 10 loci with a frequency of LOH higher than 50%. These include KDM6B (75%), MNT (71%), SMARCA2 (64%), HEC1 (60%), ANKRD5 (58%), BCL2 (58%), SEMA6D (57%), D5S818 (56%), STYK1 (50%) and BCL6B (50%) (Table 9). All but ANKRD5 and D5S818 have been associated with cancer. While LOH at chromosomal regions where KDM6B (17p13), MNT (17p13), BCL6B (17p13), HEC1 (18p11), BCL1 (18q21), ANKRD5 (20p12) and SEMA6D (15q21) reside have been observed in CRC tissues,^{24, 25} LOH at the SMARCA2 at 9p24 and STYKJ at 12p13 has not been reported in CRC carcinogens. Therefore, a possible association of LOH at the SMARCA2 with MSI-M or with LM formation was determined.

Association between LOH around the SMARCA2 locus and MSI-M in primary and LM tissues. To increase the number of informative cases for SMARCA2 LOH analysis, we used two polymorphic microsatellite markers within the SMARCA2 gene and two markers located at 230 Kb and 240 Kb away from 3′ side of the SMARCA2 gene respectively. Using the definition for the SMARCA2 region (SMARCA2R) LOH described in herein, a Korean cohort consisting of the 167 consecutive cases of primary CRC described hereinabove was analyzed. ²⁰ SMARCA2R LOH was detected in 59 of 165 (35.8%) informative cases. There was no significant association between SMARCA2R LOH and recurrent-free survival of stage II and III primary CRC by Kaplan-Meier analysis (log-rank test, P=0.205). There was also no association between SMARCA2R LOH and MSI-M in this cohort (Table 10, P=0.122). The only factor associated with SMARCA2R LOH was younger age (≦62, P=0.035).

Next, 101 cases of primary CRC that gave rise to LM for SMARCA2R LOH (Table 10) were examined. Ninety-six cases were informative for SMARCA2R LOH (FIG. 6A to 6D). Among them, 61 cases were stage II/III and 22 cases (36.1%) were positive for SMARCA2R LOH. Thirty-five cases were stage IV CRC and 14 cases (40%) were positive for SMARCA2R LOH. A significant association between SMARCA2R LOH and MSI-M was detected in stage IV CRC that gave rise to LM (P=0.017) but not in stage II/III primary CRC that gave rise to LM (P=0.811). There was also a significant association between SMARCA2R LOH and stage IV tissues collected from Korea in contrast to the tissues from Japan.

TABLE 10
SMARCA 2R LOH in primary CRC that gave rise to LM.

| | Stage II/III | | | | | Stage IV | | | | |
|---|---|---|---|---|---|---|---|---|---|---|
| Factors | LOH: n (%) | | non-LOH: n (%) | | P values^{a} | LOH: n (%) | | non-LOH: n (%) | | P values |
| Age | | | | | | | | | | |
| ≦62 | 14 | (63.6) | 21 | (53.8) | | 8 | (57.1) | 11 | (52.4) | |
| >62 | 8 | (36.4) | 18 | (46.2) | 0.214 | 6 | (42.9) | 10 | (47.6) | 0.782 |
| Sex | | | | | | | | | | |
| F | 9 | (40.9) | 13 | (33.3) | | 5 | (35.7) | 9 | (42.9) | |
| M | 13 | (59.1) | 26 | (66.7) | 0.554 | 9 | (64.3) | 12 | (57.1) | 0.673 |
| Grade^{b} | | | | | | | | | | |
| G1 | 8 | (36.4) | 13 | (33.3) | | 2 | (14.3) | 4 | (19.0) | |
| G2 + G3 | 14 | (63.3) | 26 | (66.7) | 0.811 | 12 | (85.70 | 17 | (81.0) | 0.714 |
| Location^{c} | | | | | | | | | | |
| Proximal | 4 | (18.2) | 3 | (7.7) | | 2 | (14.3) | 6 | (28.6) | |
| Distal | 18 | (81.8) | 36 | (92.3) | 0.217 | 12 | (85.7) | 15 | (71.4) | 0.324 |
| MS1 | | | | | | | | | | |
| non-MSI-M | 8 | (36.4) | 13 | (33.3) | | 5 | (35.7) | 16 | (76.2) | |
| MSI-M | 14 | (63.6) | 26 | (66.7) | 0.811 | 9 | (64.3) | 5 | (23.8) | 0.017 |
| Population | | | | | | | | | | |
| Japan | 5 | (22.7) | 7 | (17.9) | | 1 | (7.1) | 10 | (47.6) | |
| Korea | 17 | (77.3) | 32 | (82.1) | 0.652 | 13 | (92.9) | 11 | (52.4) | 0.012 |
| Total | 22 | (36.1) | 39 | (63.9) | | 14 | (40.0) | 21 | (60.0) | |

^{a}P values were determined by chi square test.
^{b}G1: well differentiated, G2: moderatly differentiated, G3: poorly differentiated.
^{c}A location of primary CRCs from which the LMs originated. Proximal includes cecum ascending and traverse colon. Distal includes sigmoid colon and rectal.

Next, 74 cases of LM for SMARCA2R LOH (Table 11, FIG. 6A to 6D) were examined. In total, 71 cases were informative for SMARCA2R LOH analysis. Among them, 39 cases were metachronous LM and 26 cases (61.5%) were positive for SMARCA2R LOH. Thirty-two cases were synchronous LM and 18 cases (56.3%) were positive for SMARCA2R LOH. A significant association between SMARCA2R LOH and MSI-M was detected in both metachronous (P=0.002) and synchronous LM (P=0.011) (Table 11).

TABLE 11
SMARCA 2R in LM.

| | Metachronous LM | | | | | Synchronous LM | | | | |
|---|---|---|---|---|---|---|---|---|---|---|
| Factors | LOH: n (%) | | non-LOH: n (%) | | P values^{a} | LOH: n (%) | | non-LOH: n (%) | | P values |
| Age | | | | | | | | | | |
| ≦62 | 14 | (58.3) | 10 | (66.7) | | 7 | (38.9) | 6 | (42.9) | |
| >62 | 10 | (41.7) | 5 | (33.3) | 0.603 | 11 | (61.1) | 8 | (57.1) | 0.821 |
| Sex | | | | | | | | | | |
| F | 6 | (25.0) | 7 | (46.7) | | 6 | (33.3) | 5 | (35.7) | |
| M | 18 | (75.0) | 8 | (53.3) | 0.163 | 12 | (66.7) | 9 | (64.3) | 0.888 |
| Grade^{b} | | | | | | | | | | |
| G1 | 11 | (45.8) | 6 | (40.0) | | 2 | (11.1) | 2 | (14.3) | |
| G2 + G3 | 13 | (54.2) | 9 | (60.0) | 0.721 | 16 | (88.9) | 12 | (85.7) | 0.788 |
| Location^{c} | | | | | | | | | | |
| Proximal | 1 | (4.2) | 2 | (13.3) | | 4 | (22.2) | 2 | (14.3) | |
| Distal | 23 | (95.8) | 13 | (86.7) | 0.296 | 14 | (77.8) | 12 | (85.7) | 0.568 |
| MSI | | | | | | | | | | |
| non-MSI-M | 3 | (12.5) | 9 | (60.0) | | 6 | (33.3) | 11 | (78.6) | |
| MSI-M | 21 | (87.5) | 6 | (40.0) | 0.002 | 12 | (66.7) | 3 | (21.4) | 0.011 |
| Population | | | | | | | | | | |
| Japan | 10 | (41.7) | 6 | (40.0) | | 5 | (27.8) | 7 | (50.0) | |
| Korea | 14 | (58.3) | 9 | (60.0) | 0.918 | 13 | (72.2) | 7 | (50.0) | 0.198 |
| Total | 24 | (61.5) | 15 | (38.5) | | 18 | (56.3) | 14 | (43.7) | |

^{a}P values were determined by chi square test.
^{b}G1: well differentiated, G2: moderatly differentiated, G3: poorly differentiated.
^{c}A location of primary CRCs from which the LMs originated. Proximal includes cecum ascending and traverse colon. Distal includes sigmoid colon and rectal.

The results above indicate that SMARCA2R LOH frequently occurred in MSI-M positive stage IV primary CRC, synchronous LM and metachronous LM compared to non-MSI-M tumor types. To further confirm these results, we performed multivariable logistic regression analysis to evaluate a significant association of MSI-M with various factors including SMARCA2R LOH. As shown in Table 12, SMARCA2R-LOH was significantly associated with MSI-M in stage IV CRC that gave rise to LM (O. R.: 9.36, 95% CI: 1.2-73.1, P=0.033) but not with stage II/III primary CRC that gave rise to LM (P=0.731). SMARCA2R LOH was also significantly associated with MSI-M in metachronous LM (O.R.: 45.6, 95% CI: 3.5-595.4, P=0.004) and synchronous LM (O.R.: 9.74, 95% CI: 1.6-59.7, P=0.014).

TABLE 12
Association between MSI-M and SMARCA 2R in primary CRC and LM.

| | Provbability of association with MSI-M (p value) | | | |
|---|---|---|---|---|
| Factors | Stage II/III^{b} | Stage IV^{c} | Metachronous LM^{d} | Synchronous LM^{e} |
| SMARCA 2R LOH | | | | |
| yes vs no | 0.731 | 0.033 | 0.004 | 0.014 |
| | | (O.R.: 9.36, | (O.R.: 45.6, | (O.R.: 9.7, |
| | | 95% CI: 1.2-73.1) | 95% CI: 3.5-595.4) | 95% CI: 1.6-59.7) |
| Age ≦62 vs >62 | 0.667 | 0.053 | 0.421 | 0.169 |
| Male vs female | 0.141 | 0.376 | 0.553 | 0.516 |
| Grade^{f} G2 + G3 vs G1 | 0.696 | 0.799 | 0.079 | 0.533 |
| Location^{g} distal vs proximal | 0.5 | 0.865 | 0.998 | 0.848 |
| Japan vs Korea | 0.413 | 0.943 | 0.959 | 0.961 |

^{a}Multivariable logistic regression analysis was performed. P values were determined by chai square test. The P values underlined were significant (<0.05) and O.R. and 95% CI values were added below them.
^{b}61 cases that gave rise to LM were analyzed
^{c}35 cases that gave rise to LM were analyzed.
^{d}32 cases were analyzed
^{e}39 cases were analyzed.
^{f}G1: well differentiated, G2: moderatly differentiated, G3: poorly differentiated.
^{g}Proximal includes cecum, ascending and traverse colon. Distal includes sigmoid colon and rectal. O.R.: Odds Ratio.

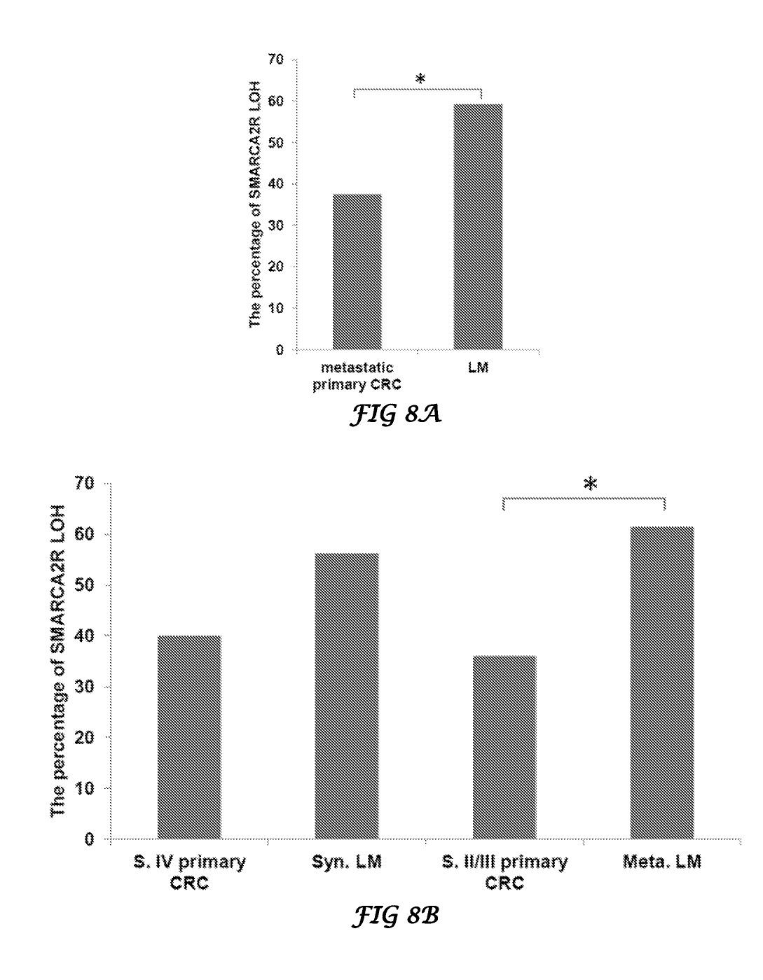

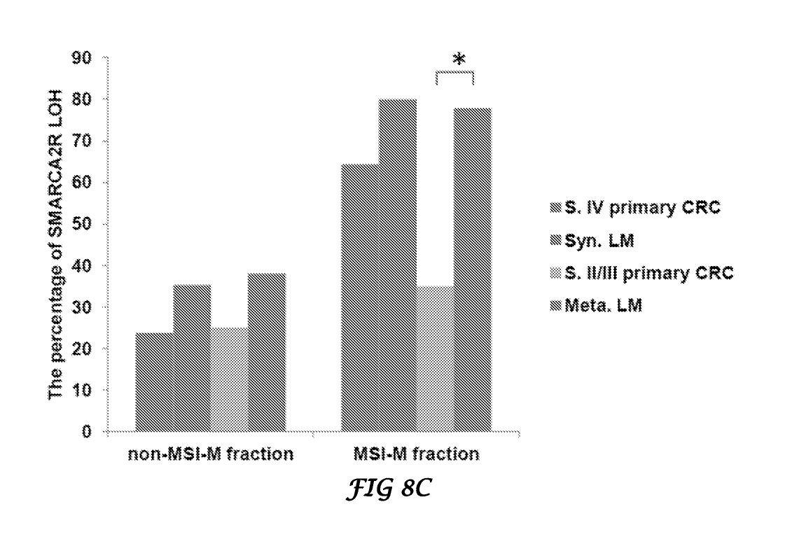

The results above also indicate that there was a significant difference in the frequency of SMARCA2R-LOH in LM tissues (59.2%, 42 of 71 cases) compared to primary CRC tissues that gave rise to LM (37.5%, 36 of 96 cases) (FIG. 8A, P=0.006). This difference was largely due to a difference between metachronous LM and metastatic stage II/III primary CRC (P=0.013) but not between synchronous LM and stage IV primary CRC (P=0.183) (FIG. 8B). Moreover, a significant difference in the frequency of SMARCA2R-LOH was observed between the MSI-M−positive fraction of metastatic stage II/III primary CRC and that of metachronous LM (P=0.001) but not between the non-MSI-M fraction of stage II/III primary and that of metachronous LM (P-0.443) (FIG. 8C). There was no significant difference in the frequency of SMARCA2R-LOH between the MSI-M-positive fraction of stage IV primary CRC and that of synchronous LM (P═), or between the non-MSI-M fraction of stage IV primary CRC (23.8%) and that of synchronous LM (35.3%) (P=0.438) (FIG. 8C).

Taken together, these results suggest that SMARCA2R-LOH plays a critical role in the formation of LM in conjunction with events associated with MSI-M. In stage IV primary CRC that is associated with synchronous LM, a high percentage of MSI-M tumors gained SMARCA2R-LOH (64.3%, FIG. 8C). These results suggest that CRC tissue that has gained MSI-M and SMARCA2R-LOH simultaneously in the early stage of tumor formation may develop synchronous LM. On the other hand, MSI-M-positive CRC that gains SMARCA2R-LOH after dissemination may develop metachronous LM. Alternatively, MSI-M and SMARCA2R-LOH double positive cells present as a minor population in the primary tissues may develop metachronous LM if not eradicated through surgery and/or chemotherapy.

It was found that LOH at the region near the SMARCA2 locus on 9p24.3 co-exists with MSI-M at high frequency in LM and stage IV primary tissues associated with synchronous LM. In contrast, SMARCA2R-LOH is less frequent in stage II/III primary CRC even in primary CRC that gave rise to LM. Furthermore, a significant difference in frequency of SMARCA2R-LOH was detected between the MSI-M fraction of stage II/III primary CRC that gave rise to LM and that of metachronous LM. These results indicate that MSI-M and SMARCA2R-LOH are genetic markers for liver metastasis from primary CRC, and suggest that a putative critical event associated with MSI-M and allelic loss of a critical gene around SMARCA2 locus cooperate to form LM from primary CRC.

It was demonstrated hereinabove that MSI-M, H-MSS and MSI-H primary CRC at stage II and III exhibited the highest, modest and lowest risks for recurrent distant metastasis respectively.²⁰ These results demonstrate that the mechanism that defines MSI-H or MSI-M can also be involved in the process that determines the probability of future recurrence. In MSI-H cases, the evidence indicated that a defective MMR that causes MSI-H may also results in increased immunogenicity and/or apoptotic potential of tumor cells through hypermutation of the genes involved in these processes, leading to a good prognosis.²⁶

Down-regulation of MSH3 may induce MSI-M in tissue cultured cell lines.¹⁶ The expression of MSH3 in MSI-M primary CRC tissues monitored by IHC was quantitatively reduced and heterogenous within these tissues compared to H-MSS primary CRC. ¹⁶ Also, some MSH3-negative tumor cells were seen near necrotic areas in MSI-M tumor tissue. These observations may indicate that down-regulation of MSH3 in CRC tissues may not be due to genetic causes but rather to physiological causes affected by microenvironmental factors, such as hypoxia.¹⁶ Furthermore, the down-regulation of MSH3 in 8 out of 10 human cell lines that were placed under hypoxia (0.1% O₂) (unpublished data) was observed. It has been reported that hypoxia down-regulates MMR genes including MSH2, MSH6, MSH3, and MLH1 and induces MSI in certain cases. ²⁷⁻²⁹ Finally, MSI-M CRC tissues with a reduced level of MSH3 over-express glucose transporter 1 protein that is a marker of hypoxia (unpublished data).³⁰ Thus, hypoxia may cause down-regulation of MSH3 in CRC tissues, leading to MSI-M. Because intra-tumor hypoxia is also known to enhance aggressiveness of cancer and promote the metastatic potential of primary tumor tissues,^{31, 32} hypoxia may be what induces MSI-M through down-regulation of MSH3 and causes critical changes that promote metastasis.

Considering a genetic mechanism of LOH for tumorigenesis, it is reasonable to assume that a gene residing around the SMARCA2R may be recessive and negatively regulate metastasis. If this is the case, there must be a first hit that inactivates one of the alleles before loss of a second normal allele. One possibility is that a putative critical event associated with MSI-M could be inactivation of the first allele at this locus through down-regulation of MSH3 or by another mechanism induced by hypoxia. These cells become competent for distant metastasis when the second hit, SMARCA2R-LOH, occurs. Alternatively, hypoxic cells may gain a change in another gene locus that may cooperate with SMARCA2R-LOH for metastasis. In conclusion, the present inventors found that SMARCA2R-LOH to be a critical genetic marker associated with MSI-M and ˜50% of LM from primary CRC.

It was found that SMARCA2R-LOH and MSI-M frequently coexist in stage IV primary CRC and LM tissues, suggesting that two events associated with these genetic changes may play a critical role for liver metastasis and be involved in liver metastasis in at least 50% of cases.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

### Example 1

U.S. Patent Application Publication No. 2011/0039272: Gene Expression Markers for Colorectal Cancer Prognosis.
U.S. Pat. No. 7,871,769: Gene Expression Markers for Predicting Response to Chemotherapy.
(1) Haugen et al. Cancer Res 2008; 68: 8465-72; 2) Yamada et al. Oncol Rep 2010; 23: 551-61.
(2) Xicola P M et al. L Natl Cancer Inst 2007; 93: 244-252.
(3) Goel A et al. PLos One 2010; 5:e9393.
(4) Boland C R et al. Cancer Res 1998; 58: 5248-5257.
(5) Halling K C et al. J Natl Cancer Inst 1999; 91:1295-1303.
(6) Ribic C M et al. N Engl J Med 2003; 349:247-257.
(7) Mori Y et al. Cancer Res 2003; 63,4577-4582.
(8) Halford S et al. Cancer Res 2002; 62: 53-57.
(9) Laiho P et al Cancer Res 2002; 62: 1166-1170.
(10) Yamada K et al. Oncol Rep 2010; 23:551-561.

### Example 2

1. Umar A, Kunkel T A. DNA-replication fidelity, mismatch repair and genome instability in cancer cells. Eur J Biochem 1996; 238: 297-307.
2. Boland C R, Thibodeau S N, Hamilton S R et al. A National Cancer Institute workshop on microsatellite instability for cancer detection and familial predisposition: Development of international criteria for the determination of microsatellite instability in colorectal cancer. Cancer Res 1998; 58: 5248-5257.
3. Xicola R M, Lior X, Pons E, et al. Performance of different microsatellite marker panels for detection of mismatch repair-deficient colorectal tumors. Natl Cancer Inst 2007; 93: 244-252.
4. Goel A, Nagasaka T, Hamelin R, et al. An optimized pentaplex PCR for detecting DNA mismatch repair-deficient colorectal cancers. PLos One 2010; 5:e9393.
5. Herman J G, Umar A, Polyak K, et al. Incidence and functional consequences of hMLH1 promoter hypermethylation in colorectal carcinoma. Proc Natl Acad Sci USA 1998; 95: 6870-5.
6. Halling K C, French Aj, McDonnell S K, et al. Microsatellite instability and 8p allelic imbalance in stage B2 and C colorectal cancers. Natl Cancer Inst 1999; 91:1295-1303.
7. Ribic C M, Sargent D J, Moore M J, et al. Tumor microsatellite-instability status as a predictor of benefit from fluorouracil-based adjuvant chemotherapy for colon cancer. N Engl J Med 2003; 349: 247-257.
8. Sinicrope F A, Foster N R, Thibodeau S N, et al. DNA mismatch repair status and colon cancer recurrence and survival in clinical trials of 5-fluoruracil-based adjuvant therapy. Natl Cancer Inst 2011; 103: 863-844.
9. Halford S, Sasieni P, Rowan A, et al. Low-level microsatellite instability occurs in most colorectal cancers and is a nonrandomly distributed quantitative trait. Cancer Res. 2002; 62:53-7.
10. Laiho P, Launonen V, Lahermo P, et al. Low-level microsatellite instability in most colorectal carcinomas. Cancer Res. 2002; 62:1166-70.
11. Mori Y, Selaru F M, Sato F, et al. The impact of microsatellite instability on the molecular phenotype of colorectal tumors. Cancer Res 2003; 63:4577-4582.
12. Kohonen-Corish M R J, Daniel J J, Chan C, et al. Low microsatellite instability is associated with poor prognosis in stage C colon cancer. J Clin Oncol 2005; 23:2318-2324.
13. Wright C M, Dent OF, Newland R C, et al. Low level microsatellite instability may be associated with reduced cancer specific survival in sporadic stage C colorectal carcinoma. Gut 2005; 54:103-108.
14. Mao L, Lee D J, Tockman M S, Erozan Y S, Askin F, Sidransky D. Microsatellite alterations as clonal markers for the detection of human cancer. Proc Natl Acad Sci USA 1994; 91: 9871-9875.
15. Bacolla A, Larson J E, Collins J R, et al. Abundance and length of simple repeats in vertebrate genomes are determined by their structural properties. Genome Res 2008; 18:1545-1553.
16. Haugen A, Goel A, Yamada K et al. Genetic instability caused by loss of MutS homologue 3 in human colorectal cancer. Cancer Res 2008; 68: 8465-8472.
17. Yamada K, Kanazawa S, Koike j, et al. Microsatellite instability at tetranucleotide repeats in sporadic colorectal cancer in Japan. Oncol Rep 2010; 23:551-561.
18. Lee S Y, Chung H, Devaraj B, et al. Microsatellite alterations at selected tetranucleotide repeats are associated with morphologies of colorectal neoplasias. Gastroenterology 2010; 139:1519-1525.
19. Ahrendt S A, Decker P A, Doffek K, et al. Microsatellite instability at selected tetranucleotide repeats is associated with p53 mutations in non-small cell lung cancer. Cancer Res 60 (2000) 2488-2491.
20. Garcia M, Choi C, KimH-R, et al. Moderate Levels of Microsatellite Instability are Associated with Recurrent Distant Metastasis in Stage II and III Primary Colorectal Cancers. Gastroenterology 2012 (in press)
21. Schuelke M. An economic method for the florescent labeling of PCR fragments. Nat Biotech 2000; 18:233-4.
22. Kozlowski P, de Mezer M, Krzyzosiak W J. Trinucleotide repeats in human genome and exome. Nucleic Acids Res 2010; 38:4027-4039.
23. Missirlis P I, Mead C L, Butland S L, et al. Satellog: a database for the identification and prioritization of satellite repeats in disease association studies. BMC Bioinformatics 2005; 6:145.
24. Kurashina K, Yamashita Y, Ueno T, et al. Chromosome copy number analysis in screening for prognosis-related genomic regions in colorectal carcinoma. Cancer Sci 2008; 99:1835-1840.
25. Lips E H, Dierssen J W, van EijK R, et al. Reliable high-throughput genotyping and loss-of-heterozygosity detection in formalin-fixed, paraffin-embedded tumors using single nucleotide polymorphism arrays. Cancer Res 2005; 65:10188-10191.
26. Banerjea A, Bustin S A, Dorudi S. The immunogenicity of colorectal cancers with high-degree microsatellite instability. World J Surg Oncol 2005; 3:26.
27. Mihaylova V T, Bindra R S, Yuan J, et al. Decreased expression of the DNA mismatch repair gene Mlh1 under hypoxic stress in mammalian cells. Mol Cell Biol 2003; 23:3265-3273.
28. Koshiji M, To K K, Hammer S, et al. HIF-1 alpha induces genetic instability by transcriptionally downregulating MutSalpha expression, Mol Cell 2005; 17:793-803.
29. Rodriguez-Jimenez F J, Moreno-Manzano V, Lucas-Dominguez R, et al. Hypoxia causes downregulation of mismatch repair system and genomic instability in stem cells, Stem Cells 2008; 26:2052-2062.
30. De Bock K, Mazzone M, Carmeliet P. Antiangiogenic therapy, hypoxia, and metastasis: risky liaisons, or not? Nat Rev Clin Oncol 2011; 8:393-404.
31. Chung F Y, Huang M Y, Yeh C S, et al. GLUT1 gene is a potential hypoxic marker in colorectal cancer patients. BMC Cancer 9 (2009) 241.
32. Chang Q, Jurisica I, Do T, et al. Hypoxia predicts aggressive growth and spontaneous metastasis formation from orthotopically grown primary xenografts of human pancreatic cancer. Cancer Res 2011; 71:3110-3120.

What is claimed is:

1. A method for predicting probability of recurrence free survival, determining risk of recurrence, or both in a human subject suffering from primary colorectal cancer (CRC) comprising the steps of:
identifying the human subject suffering from the primary CRC;
isolating a genomic DNA from one or more biological samples obtained from the subject, wherein the biological samples are selected from the group consisting of a frozen or fresh tissue sample; a FFPE tissue; a fecal sample; one or more biological fluids; or any combinations thereof;
measuring or determining a level of at least one of a microsatellite instability (MSI) at a mononucleotide repeat loci, a dinucleotide repeat loci, an elevated microsatellite alteration at selected tetranucleotide repeat (EMAST) loci, or a SMARCA2R-LOH, wherein the measurement is accomplished using a microsatellite assay or microarray comprising a marker panel of at least one marker representative of each of the mono-, di- and tetranucleotide repeat loci;
determining a presence or an absence of the MSI in the primary CRC from the isolated genomic DNA obtained from the human subject;
classifying the MSI in the primary CRC into MSI-H, MSI-M and H-MSS by using a classification scheme, wherein the classification scheme comprises:
a high level of microsatellite instability (MSI-H) phenotype indicative of a presence of MSI at three or more of the mono- or dinucleotide markers;
a low level of microsatellite instability (MSI-L) phenotype indicative of a presence of MSI at least one but no more than two of the mono- or dinucleotide markers;
a stable level of microsatellite stability (MSS) phenotype indicative no MSI at any of the mono- or dinucleotide markers;
a EMAST⁺ phenotype indicative of a non MSI-H phenotype with MSI at least one of the tetranucleotide markers;
a EMAST⁻ phenotype indicative of a non MSI-H phenotype with no MSI at any of the tetranucleotide markers;
a moderate level of microsatellite instability (MSI-M) phenotype indicative of a MSI-L or EMAST or both MSI-L and EMAST⁺ phenotype; and
a highly stable microsatellite (H-MSS) phenotype indicative of non MSI at any of the mono-, di-, and tetranucleotide markers; and
predicting probability of recurrence free survival, determining risk of recurrence, or both after classifying the primary CRC, wherein presence of MSI-M phenotype is indicative of a highest risk for recurrent distant metastasis, presence of MSI-H phenotype is indicative of lowest risk and H-MSS phenotype is indicative of an intermediate risk for recurrent distant metastasis in the human subject.

2. The method of claim 1, wherein the mononucleotide repeat loci markers comprise BAT25, BAT26, or both.

3. The method of claim 1, wherein the dinucleotide repeat loci markers comprise D2S123; D5S346; D175250; D18564; D18569; or any combinations thereof.

4. The method of claim 1, wherein the tetranucleotide repeat loci markers comprise MYCL1; D20582; D20585; L17835; D8S321; D9S242; D195394; or any combinations thereof.

5. The method of claim 1, wherein the marker panel comprises BAT25; BAT26; D2S123; D5S346; D175250; D18564; D18569; MYCL1; D20582; D20585; L17835; D8S321; D9S242; and D195394.

6. The method of claim 1, wherein a presence of the MSI-M phenotype in stage II and III primary CRC is indicative of high risk for a recurrent distant metastasis including a liver metastasis (LM) in the human subject.

7. The method of claim 1, wherein the method is used for treating a patient suffering from colorectal cancer; selecting an anti-neoplastic agent therapy for a patient suffering from colorectal cancer; stratifying a patient in a subgroup of colorectal cancer or for a colorectal cancer therapy clinical trial; determining resistance or responsiveness to a colorectal cancer therapeutic regimen; developing a kit for diagnosis of colorectal cancer; or any combinations thereof.

8. The method of claim 1, wherein the presence of both the MSI-M and the SMARCA2R-LOH are indicative of liver metastasis from primary CRC.

9. A method for classifying microsatellite instability (MSI) in a primary colorectal cancer (CRC) comprising:
providing a panel comprising of mono-, di-, and tetranucleotide repeat loci markers to be used in a MSI assay, wherein the markers are selected from the group consisting of BAT25; BAT26; D2S123; D5S346; D175250; D18564; D18569; MYCL1; D20582; D20585; L17835; D8S321; D9S242; and D195394;
providing a genomic DNA isolated from one or more biological samples from a human subject suffering from or the CRC;
determining a presence or an absence of the MSI in the primary CRC from the isolated genomic DNA obtained from the human subject; and
classifying the MSI or determining a tumor phenotype based on a scheme, wherein the scheme comprises:
a MSI-H phenotype indicative of a presence of MSI at three or more of the mono- or dinucleotide markers;
a MSI-L phenotype indicative of a presence of MSI at least one but no more than two of the mono- or dinucleotide markers;
a MSS phenotype indicative no MSI at any of the mono- or dinucleotide markers;
a EMAST⁺ phenotype indicative of a non MSI-H phenotype with MSI at least one of the tetranucleotide markers;
a EMAST⁻ phenotype indicative of a non MSI-H phenotype with no MSI at any of the tetranucleotide markers;
a MSI-M phenotype indicative of a MSI-L, EMAST, or both MSI-L and EMAST phenotype; and
a H-MSS phenotype indicative of non MSI at any of the mono-, di-, and tetranucleotide markers.

10. The method of claim 9, wherein the method further comprises detecting the presence of a SMARCA2R-LOH, wherein the presence of both a MSI-H and SMARCA2R-LOH are indicative of liver metastasis from primary CRC.

11. The method of claim 9, wherein the method is used to predicting probability of recurrence free survival; determining risk of recurrence; determining a stage of cancer metastasis; risk for a liver metastasis (LM); or any combinations thereof in the human subject.

12. The method of claim 9, wherein the method is used for treating a patient suffering from colorectal cancer; selecting an anti-neoplastic agent therapy for a patient suffering from colorectal cancer; stratifying a patient in a subgroup of colorectal cancer or for a colorectal cancer therapy clinical trial; determining resistance or responsiveness to a colorectal cancer therapeutic regimen; developing a kit for diagnosis of colorectal cancer; or any combinations thereof.

13. A biomarker for predicting probability of recurrence free survival; determining risk of recurrence; determining risk for a liver metastasis (LM); or any combinations thereof, in a human subject suffering from or suspected of suffering from primary colorectal cancer (CRC) comprising detection of a microsatellite alterations at a tetranucleotide repeat (EMAST), a low levels of dinucleotide repeat loci (MSI-L), or both in the sample, wherein a presence of a MSI-M or a MSI-M and a SMARCA2R-LOH phenotype in a majority of cells in a sample from stage II and III CRC subject is indicative of a high risk for recurrence, a high risk for liver metastasis (LM), or any combinations thereof in the human subject.

14. The biomarker of claim 11, wherein a determination of a MSI-M phenotype in the cells is based on a panel comprising mono-, di-, and tetranucleotide repeat loci markers.

15. The biomarker of claim 11, wherein the panel comprises BAT25; BAT26; D2S123; D5S346; D17S250; D18S64; D18S69; MYCL1; D20S82; D20S85; L17835; D8S321; D9S242; and D19S394.

16. The biomarker of claim 11, wherein the SMARCA2R-LOH phenotype is determined using the nucleic acids of SEQ ID NOS: 1 to 6.

17. A kit for predicting probability of recurrence free survival, determining risk of recurrence, or both in a human subject suffering from primary colorectal cancer (CRC) comprising:
biomarker detecting reagents for measuring a microsatellite instability (MSI) at a tetranucleotide repeat (EMAST), A mono- or dinucleotide repeat loci (MSI-L), or a SMARCA2R-LOH in a biological sample from a subject; and
instructions for predicting probability of recurrence free survival, determining risk of recurrence, or both, wherein the instructions comprise step-by-step directions for determining presence of a MSI-M, MSI-H, H-MSS or a SMARCA2R-LOH phenotype in the biological sample obtained from a subject suffering from stage II or III CRC and comparing it with the biological obtained from a normal tissue from the same subject.

18. The kit of claim 17, wherein the detecting reagents detect one or more mononucleotide, dinucleotide, or tetranucleotide repeat loci markers selected from the group consisting of BAT25; BAT26; D2S123; D5S346; D175250; D18564; D18569; MYCL1; D20S82; D20S85; L17835; D8S321; D9S242; and D195394.

19. The kit of claim 17, wherein the presence of a MSI-M phenotype or the MSI-M and SMARCA2R-LOH phenotype in a majority of cells in the sample from the subject is indicative of a high risk for recurrence and a lowered probability of recurrence-free survival in the human subject.

20. The kit of claim 17, wherein a presence of the MSI-M phenotype in the one or more cells is indicative of a high risk for liver metastasis (LM) in the subject.

21. The kit of claim 17, wherein the biological samples are selected from the group consisting of a frozen or fresh tissue sample, a FFPE tissue sample, a biopsy, a fecal sample, one or more biological fluids, or any combinations thereof.

22. The kit of claim 17, wherein the SMARCA2R-LOH is determined using SEQ ID NOS: 1 to 6.

23. A method for predicting probability of success of the cancer therapy in a patient diagnosed with primary colorectal cancer (CRC), the method comprising:
identifying the patient diagnosed with the primary CRC; and
determining a level of microsatellite instability (MSI) at one or more mononucleotide, dinucleotide, tetranucleotide repeats (EMAST), or any combinations thereof in cells obtained from one or more biological samples from the patient, wherein a presence of a MSI-M phenotype in a majority of cells in a sample from the stage II or III CRC subject is indicative of a high risk for recurrence, a high risk for liver metastasis (LM), a lowered possibility of success with the cancer therapy or any combinations thereof.

24. The method of claim 23, wherein the step of determining the MSI further comprises the steps of:
providing a panel comprising of mono-, di-, and tetranucleotide repeat loci markers to be used in a MSI assay, wherein the markers are selected from the group consisting of BAT25; BAT26; D2S123; D5S346; D175250; D18564; D18569; MYCL1; D20S82; D20S85; L17835; D8S321; D9S242; and D195394; or SMARCA2R-LOH;
providing a genomic DNA isolated from one or more biological samples from the patient diagnosed with the CRC;
determining a presence or an absence of the MSI in the primary CRC from the isolated genomic DNA obtained from the human subject; and
classifying the MSI or determining the tumor phenotype based on a scheme, wherein the scheme comprises:
a MSI-H phenotype indicative of a presence of MSI at three or more of the mono- or dinucleotide markers;
a MSI-L phenotype indicative of a presence of MSI at least one but no more than two of the mono- or dinucleotide markers;
a MSS phenotype indicative no MSI at any of the mono- or dinucleotide markers;
a EMAST⁺ phenotype indicative of a non MSI-H phenotype with MSI at least one of the tetranucleotide markers;
a EMAST⁻ phenotype indicative of a non MSI-H phenotype with no MSI at any of the tetranucleotide markers;
a MSI-M phenotype indicative of a MSI-L or EMAST or both MSI-L and EMAST phenotype; and
a H-MSS phenotype indicative of non MSI at any of the mono-, di-, and tetranucleotide markers.

25. The method of claim 23, wherein the sample is selected from the group consisting of a frozen or fresh tissue sample, a FFPE tissue sample, a fecal sample, one or more biological fluids, or any combinations thereof.

26. The method of claim 23, wherein the presence of the MSI-M, EMAST/MSI-L phenotype in the one or more cells of stage II or III CRC is indicative of metachronous liver metastasis.

27. A method for selecting a cancer therapy in a patient diagnosed with primary colorectal cancer (CRC), the method comprising:
identifying the patient diagnosed with the primary CRC;
determining a level of microsatellite instability (MSI) at one or more mononucleotide, dinucleotide, tetranucleotide repeats (EMAST), or any combinations thereof in cells obtained from one or more biological samples from the patient, wherein a presence of a MSI-M phenotype in a majority of cells in a sample from the stage II or III CRC subject is indicative of a high risk for recurrence, a high risk for liver metastasis (LM), a lowered possibility of success with the cancer therapy or any combinations thereof and presence of a H-MSS phenotype is indicative of a high probability for recurrence-free survival in the human subject; and
selecting the cancer therapy based on identifying agents to lower or suppress the MSI-M, MSS phenotype.

28. The method of claim 27, wherein the step of determining the MSI further comprises the steps of:
providing a panel comprising of mono-, di-, and tetranucleotide repeat loci markers to be used in a MSI assay, wherein the markers are selected from the group consisting of BAT25; BAT26; D2S123; D5S346; D17S250; D18S64; D18S69; MYCL1; D20S82; D20S85; L17835; D8S321; D9S242; and D19S394;
providing a genomic DNA isolated from one or more biological samples from the patient diagnosed with the CRC;
determining a presence or an absence of the MSI in the primary CRC from the isolated genomic DNA obtained from the human subject; and
classifying the MSI or determining the tumor phenotype based on a scheme and categorizing CRC into 3 groups including MSI-H, MSI-M and H-MSS, wherein the scheme comprises:
a MSI-H phenotype indicative of a presence of MSI at three or more of the mono- or dinucleotide markers;
a MSI-L phenotype indicative of a presence of MSI at least one but no more than two of the mono- or dinucleotide markers;
a MSS phenotype indicative no MSI at any of the mono- or dinucleotide markers;
a EMAST⁺ phenotype indicative of a non MSI-H phenotype with MSI at least one of the tetranucleotide markers;
a EMAST⁻ phenotype indicative of a non MSI-H phenotype with no MSI at any of the tetranucleotide markers;
a MSI-M phenotype indicative of a MSI-L or EMAST or both MSI-L and EMAST phenotype; and
a H-MSS phenotype indicative of non MSI at any of the mono-, di-, and tetranucleotide markers.

29. The method of claim 27, wherein the method further comprises detecting the presence of a SMARCA2R-LOH, wherein the presence of both a MSI-H and SMARCA2R-LOH are indicative of liver metastasis from primary CRC.

30. The method of claim 27, wherein the sample is selected from the group consisting of a frozen or fresh tissue sample, a FFPE tissue sample, a fecal sample, a cell homogenate, one or more biological fluids, or any combinations thereof.

31. A method of performing a clinical trial to evaluate a candidate drug believed to be useful in treating colorectal liver metastasis, promoting recurrence-free survival, or both, the method comprising:
a) determining a level of microsatellite instability at least one of one or more tetranucleotide repeats (EMAST), a mono- and dinucleotide repeat loci (MSI-L), or a SMARCA2R-LOH, in cells obtained from a patient, wherein a MSI-M phenotype in a majority of cells in a sample from the patient is indicative of a highest risk for recurrence, a high risk for liver metastasis (LM), or any combinations thereof and presence of MSI-H phenotype is indicative of lowest risk and H-MSS phenotype is indicative of an intermediate risk for recurrent distant metastasis;
b) administering a candidate drug to a first subset of the patients, and
a placebo to a second subset of the patients;
a comparator drug to a second subset of the patients; or
a drug combination of the candidate drug and another active agent to a second subset of patients;
c) repeating step a) after the administration of the candidate drug or the placebo, the comparator drug or the drug combination; and
d) monitoring a recurrent-free survival rate exhibited by stage II and III primary CRC patients with an MSI-H, an MSI-M, or an H-MSS phenotype that is statistically significant as compared to the rate exhibited by the patients with the MSI-H, the MSI-M, the H-MSS and the SMARCA2R-LOH, phenotypes occurring in the second subset of patients, wherein a statistically significant increase indicates that the candidate drug is useful in treating said disease state.

32. A method for determining the risk for development of colorectal liver metastasis in a human subject suffering from colorectal cancer (CRC) comprising the steps of:
identifying the human subject suffering from the primary CRC;
obtaining one or more biological samples from the subject, wherein the biological samples are selected from the group consisting of a frozen or fresh tissue sample, a FFPE tissue sample, a fecal sample, one or more biological fluids, or any combinations thereof;
measuring or determining a level of a microsatellite instability (MSI) using a microsatellite assay comprising a panel of a mononucleotide repeat loci, a dinucleotide repeat loci, and a tetranucleotide (EMAST) repeat loci selected from the group consisting of BAT25; BAT26; D2S123; D5S346; D17S250; D18S64; D18S69; MYCL1; D20S82; D20S85; L17835; D8S321; D9S242; and D19S394 and a SMARCA2R-LOH;
determining a presence or an absence of the MSI in the primary CRC from the isolated genomic DNA obtained from the human subject;
classifying the MSI in the primary CRC by using a classification scheme, wherein the classification scheme comprises:
a MSI-H phenotype indicative of a presence of MSI at three or more of the mono- or dinucleotide markers;
a MSI-L phenotype indicative of a presence of MSI at at least one but no more than two of the mono- or dinucleotide markers;
a MSS phenotype indicative no MSI at any of the mono- or dinucleotide markers;
a EMAST⁺ phenotype indicative of a non MSI-H phenotype with MSI at at least one of the tetranucleotide markers;
a EMAST⁻ phenotype indicative of a non MSI-H phenotype with no MSI at any of the tetranucleotide markers;
a MSI-M phenotype indicative of a MSI-L or EMAST or both MSI-L and EMAST phenotype; and
a H-MSS phenotype indicative of non MSI at any of the mono-, di-, and tetranucleotide markers; and
determining the risk for colorectal cancer liver metastasis in the human subject based on a presence or an increase in the MSI-M phenotype in the sample.

33. The method of claim 32, wherein the presence of the MSI-M phenotype in the stage II and III primary CRC sample is predictive of metachronous liver metastasis.

34. The method of claim 32, wherein the presence of both the SMARCA2R-LOH and the MSI-M are indicative of stage IV primary CRC and LM.

35. The method of claim 32, wherein the method is used for treating a patient suffering from colorectal cancer, selecting an anti-neoplastic agent therapy for a patient suffering from colorectal cancer, stratifying a patient in a subgroup of colorectal cancer or for a colorectal cancer therapy clinical trial, determining resistance or responsiveness to a colorectal cancer therapeutic regimen, developing a kit for diagnosis of colorectal cancer, or any combinations thereof.


SEQUENCE LISTING (8 sequences)

SEQ ID NO: 1 - DNA, 36 bp, artificial sequence
  synthetic oligonucleotide
tgtaaaacgacggccagtaggggaaaaggacgttgc

SEQ ID NO: 2 - DNA, 19 bp, artificial sequence
  synthetic oligonucleotide
tgttgttgctgcgtctgtg

SEQ ID NO: 3 - DNA, 40 bp, artificial sequence
  synthetic oligonucleotide
tgtaaaacgacggccagtagcctgaacactgcatagtgag

SEQ ID NO: 4 - DNA, 24 bp, artificial sequence
  synthetic oligonucleotide
tcatcttttggaaatggaataagg

SEQ ID NO: 5 - DNA, 24 bp, artificial sequence
  synthetic oligonucleotide
gaaacataaccaagaagatggatg

SEQ ID NO: 6 - DNA, 38 bp, artificial sequence
  synthetic oligonucleotide
tgtaaaacgacggccagtccagcttctgcaatggtgta

SEQ ID NO: 7 - DNA, 22 bp, artificial sequence
  synthetic oligonucleotide
tttttaaacagcccaactttca

SEQ ID NO: 8 - DNA, 20 bp, artificial sequence
  synthetic oligonucleotide
cacacccacttttcagagga